(19) United States
(12) Patent Application Publication
(10) Pub. No.: US 2026/0327676 A1
(43) Pub. Date: Oct. 22, 2026

(54) CHEMILUMINESCENT REAGENTS FOR DETECTION OF ALKALINE PHOSPHATASES

(71) Applicant: BECKMAN COULTER, INC., Brea, CA (US)

(72) Inventors: Guoping Wang, Brea, CA (US); Khaledur Rashid, Brea, CA (US); Humayun Kabir, Brea, CA (US); Barry A. Schoenfelner, Brea, CA (US); Christopher R. Knutson, Brea, CA (US); Ross Conn, Brea, CA (US)

(21) Appl. No.: 18/998,708

(22) PCT Filed: Jul. 28, 2023
(86) PCT No.: PCT/US2023/071231

§ 371 Date: Jan. 27, 2025

Related U.S. Application Data
(60) Provisional application No. 63/369,879, filed on Jul. 29, 2022.

(51) Int. Cl. C12Q 1/42; C09K 11/02; C09K 11/07; G01N 21/76

(52) U.S. Cl. CPC C12Q 1/42; C09K 11/025; C09K 11/07; C12Y 301/03001; G01N 21/76; C09K 2211/1007; C09K 2211/1018

(57) ABSTRACT

Described herein are 1,2-dioxetanes that are useful as chemiluminescent probes, diagnostic agents, and imaging agents. Also described herein are compositions containing such compounds and methods of using the same.

### CROSS-REFERENCE TO RELATED APPLICATIONS

This application claims the benefit of U.S. Provisional Appl. No. 63/369,879, filed Jul. 29, 2022, which is incorporated by reference as if fully set forth herein.

### BACKGROUND

Chemiluminescent dioxetanes are strained cyclic peroxides that can undergo rapid decomposition to generate an excited, transient species that subsequently decays to ground state via emission of light.

Such compounds are useful as luminescent probes in a range of assays, including enzyme activity assays, immunoassays, and DNA detection assays. Chemiluminescence-based assays can offer excellent sensitivity because, unlike fluorescence and absorption-based assays, no light excitation is required.

Dioxetanes can be generated in situ at the time of their use or prepared in advance in stable form and then later activated. When generated in situ via oxidation of a precursor alkene, chemiluminescent dioxetanes can also function as a detection or imaging method for reactive oxygen species (ROS). An example of a stable chemiluminescent dioxetane is 4-methoxy-4-(3-phosphatephenyl)spiro[1,2-dioxetane-3,2′-adamantane]. This compound, also known as LUMIGEN® PPD, can be activated upon treatment with alkaline phosphatase (ALP). ALP is an enzyme that catalyzes the hydrolysis of phosphate groups. Once activated, the resulting compound subsequently undergoes fragmentation of the 1,2-dioxatane ring and emits light, thus functioning as a luminescent probe in ALP-labeled assays.

Dioxetane compounds have been developed that are sensitive and strongly emissive under non-aqueous conditions. However, such compounds suffer from weak emissions in aqueous media and may take a long time to reach maximum luminescence after contact with a desired analyte.

### SUMMARY

There is a need for dioxetanes that provide a rapid response to the presence of an analyte and the concentration of an analyte, or both. There is also a need for dioxetanes that are strongly emissive and suitable for use in aqueous environments. This disclosure describes compositions that include a combination of a dioxetane and one or more selected surfactants that provide unexpectedly improved emission results in aqueous environments. Certain combinations, for example, can provide a dramatic relative difference in brightness in relative light units (RLU) (over 20 times higher) depending on the dioxetane and surfactant combination. One significant benefit of the compositions described herein is that one can improve alkaline phosphatase detection limits. For example, with the compositions described herein, one can detect alkaline phosphatase concentrations from 1×10⁻¹² mol/μL to 1×10⁻²³ mol/μL. For example in some aspects, the compositions described herein may be used to detect ALP in a composition having a ALP concentration of at least 1×10⁻²³ mol/μL, at least 1×10⁻²² mol/μL, at least 1×10⁻²¹ mol/μL, at least 1×10⁻²⁰ mol/μL, or at least 1×10⁻¹⁷ mol/μL; additionally or alternatively, the compositions described herein may be used to detect ALP in a composition having a ALP concentration of up to (that is, at most) 1×10⁻²¹ mol/μL, up to 1×10⁻²⁰ mol/μL, up to 1×10⁻¹⁹ mol/μL, up to 1×10⁻¹⁸ mol/μL, up to 1×10⁻¹⁵ mol/μL, up to 1×10⁻¹⁰ mol/μL, or up to 1×10⁻⁵ mol/μL. In some exemplary aspects, the compositions described herein may be used to detect in AP4, having an ALP concentration of 1.2×10⁻¹⁵ mol/μL; AP6, having an ALP concentration of 1.2×10⁻¹⁷ mol/μL; AP8, having an ALP concentration of 1.2×10⁻¹⁹ mol/μL; and AP9 having an ALP concentration of 1.2×10⁻²⁰ mol/μL. For example, with the compositions described herein, one can detect 1×10⁻¹¹ mol to 1×10⁻²² mol alkaline phosphatase (such as 1×10⁻¹⁴ mol, 1×10⁻¹⁵ mol, 1×10⁻¹⁶ mol, 1×10⁻¹⁷ mol, 1×10⁻¹⁸ mol, 1×10⁻¹⁹ mol, 1×10⁻²⁰ mol, 1×10⁻²¹ mol, 1×10⁻²² mol, 1×10⁻²³ mol; from 1×10⁻¹³ mol to 1×10⁻²² mol, from 1×10⁻¹¹ mol to 1×10⁻²⁰ mol, from 1×10⁻¹³ mol to 1×10⁻¹⁸ mol, from 1×10⁻¹⁵ mol to 1×10²⁰, or from 1×10⁻¹⁴ mol to 1×10⁻¹⁷ mol alkaline phosphatase), such as in a composition comprising 5 μL to 50 μL (for example, 10 μL AP9) of an alkaline phosphatase composition diluted in 50 μL to 250 μL (for example, 100 μL) buffer, such as a composition comprising from 1×10⁻¹¹ mol to 1×10⁻²² mol alkaline phosphatase.

The disclosure provides a composition comprising a compound of Formula I and salts thereof:

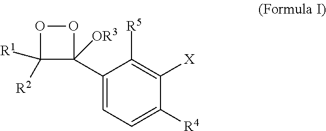

wherein each of R¹ and R² is independently C₃-C₁₀ alkyl, or R¹ and R² taken together with the carbon to which they are attached provide a C₅-C₁₀ cycloalkyl ring;
R³ is C₁-C₁₀ alkyl, C₆-C₁₀ aryl, or heteroaryl;
R⁴ is C₂-C₁₀ alkenyl;
R⁵ is H or C₁-C₁₀ alkyl;
X is a phosphate; and at least one surfactant.

The terms “surfactant” and “enhancer” are used interchangeably herein. Examples of such surfactants include, but are not limited to, phosphonium surfactants, which are also referred to herein interchangeably as “phosphonium enhancers.” Phosphonium surfactants include polymeric phosphonium surfactants and small molecule phosphonium surfactants. Examples of polymeric phosphonium surfactants include polyvinyl type polymers with pendant quaternary phosphonium groups, which are disclosed in U.S. Pat. No. 5,393,469.

Exemplary polymeric phosphonium enhancers include polyvinylbenzyltributylphosphonium chloride copolymer with polyvinylbenzyltrioctylphosphonium chloride and polyvinylbenzyltributylphosphonium chloride as well as dicationic compounds bearing two quaternary ammonium or phosphonium groups, such as those disclosed in U.S. Pat. No. 5,451,347.

Examples of polymeric phosphonium enhancers include polymers comprising repeating unit (A), repeating unit (B) or both:

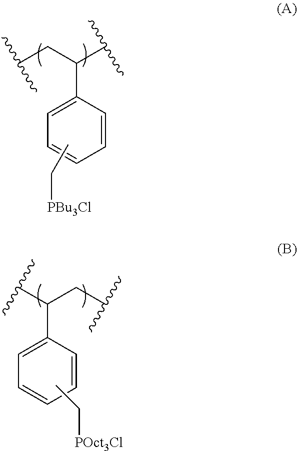

wherein “Bu₃” refers to “tributyl, “Oct₃” refers to “trioctyl.” A molar ratio of the repeating unit (A) to repeating unit (B) may be in a range of from 1:100 to 100:1 (for example, from 100:1 to 1:1, including, for example a ratio of 80:1, 70:1; 60:1, 50:1; 40:1, 30:1, 20:1, 10:1, 9:1, 8:1, 7:1, 6:1, 5:1, 4:1, 70:30, 75:25, 60:40 or 50:50). The weight average molecular weight (M_{w}) may be for example, in a range from 100,000 g/mol to 400,000 g/mol, from 150,000 g/mol to 350,000 g/mol, from 200,000 g/mol to 300,000 g/mol. For example, in exemplary aspects, the M_{w} can be 100,000, 120,000, 140,000, 160,000, 180,000, 200,000, 220,000, 240,000, 260,000, 280,000, 300,000, 320,000, 340,000, 360,000, 380,000, or 400,000 g/mol. In some embodiments, the M_{w} is 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol. The copolymers may be random copolymers, block copolymers, or random-block copolymers.

Examples of polymeric phosphonium enhancers include polymers of the general formula:

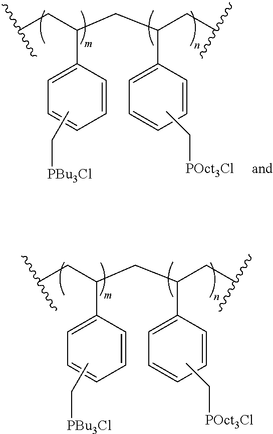

wherein m and n are each, independently, integers from 0 to 1000 (for example, 1 to 500, 250 to 1000, 300 to 900, 50 to 500 or 250 to 750). The ratio of m:n may be, for example, from 100:1 to 1:1, including, for example, a ratio of m:n of 80:1, 70:1; 60:1, 50:1; 40:1, 30:1, 20:1, 10:1, 9:1, 8:1, 7:1, 6:1, 5:1, 4:1, 70:30, 75:25, 60:40, or 50:50; m+n is in a range of 100 to 1000 (for example, m+n may be 100 to 600, 200 to 500, or 300 to 600); the weight average molecular weight (M_{w}) may be for example, in a range from 100,000 g/mol to 400,000 g/mol, from 150,000 g/mol to 350,000 g/mol, from 200,000 g/mol to 300,000 g/mol. For example, in exemplary aspects, the M_{w} can be 100,000, 120,000, 140,000, 160,000, 180,000, 200,000, 220,000, 240,000, 260,000, 280,000, 300,000, 320,000, 340,000, 360,000, 380,000, or 400,000 g/mol. In some embodiments, the M_{w} is 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol. The copolymers may be random copolymers, block copolymers, or random-block copolymers.

Exemplary polymeric phosphonium enhancers/surfactants also include polymeric phosphonium surfactants comprising the structure:

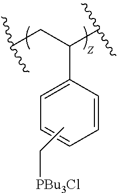

wherein z is an integer from 2 to 1000 (for example, 2 to 500, 100 to 600, 450 to 800, 300 to 600, 2 to 250, 50 to 250, 100 to 300, 150 to 400, 175 to 400, 50 to 300, 100 to 500, 150 to 400, or 10 to 200). The weight average molecular weight (M_{w}) may be for example, in a range from 500 g/mol to 500,000 g/mol, from 500 g/mol to 100,000 g/mol, 50,000 g/mol to 100,000 g/mol, from 150,000 g/mol to 350,000 g/mol or from 200,000 g/mol to 300,000 g/mol. For example, in exemplary aspects, the M_{w} can be 50,000, 60,000, 70,000, 100,000, 120,000, 140,000, 160,000, 180,000, 200,000, 220,000, 240,000, 260,000, 280,000, 300,000, 320,000, 340,000, or 350,000 g/mol. In some embodiments, the M_{w} is 70,000, 80,000, 100,000 or 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol. Examples of such polymeric phosphonium enhancers/surfactants include those comprising the structures:

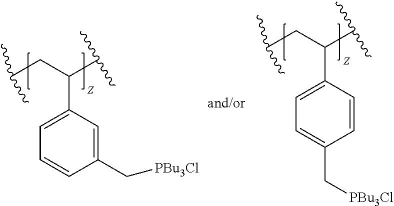

wherein z is an integer from 2 to 1000 (for example, 2 to 500, 100 to 600, 450 to 800, 300 to 600, 2 to 250, 50 to 250, 100 to 300, 150 to 400, 175 to 400, 50 to 300, 100 to 500, 150 to 400, or 10 to 200). The weight average molecular weight (M_{w}) may be for example, in a range from 500 g/mol to 500,000 g/mol, from 500 g/mol to 100,000 g/mol, 50,000 g/mol to 100,000 g/mol, from 150,000 g/mol to 350,000 g/mol or from 200,000 g/mol to 300,000 g/mol. For example, in exemplary aspects, the M_{w} can be 50,000, 60,000, 70,000, 100,000, 120,000, 140,000, 160,000, 180,000, 200,000, 220,000, 240,000, 260,000, 280,000, 300,000, 320,000, 340,000, or 350,000 g/mol. In some embodiments, the M_{w} is 70,000, 80,000, 100,000 or 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol.

Exemplary small molecule phosphonium enhancers/surfactants include small molecule surfactants of the formula:

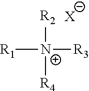

wherein:
R¹²-R¹⁴ are each, independently, C₁-C₁₀ alkyl;
R¹⁵ is arylalkyl; and
X⁻ is a counterion (for example, chloride). R¹⁵ may be arylalkyl substituted with a C₂-C₈ alkenyl group or with a group of the formula:

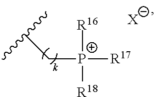

wherein k is 0, 1, or 2; R¹⁶-R¹⁸ are each, independently, C₁-C₁₆ alkyl. R¹⁶-R¹⁸ may each, independently, be C₄-C₁₀ alkyl. The C₂-C₈ alkenyl group may be a group of the formula:

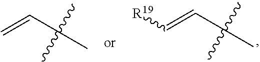

wherein R¹⁹ is C₁-C₈ alkyl, C₂-C₈ alkenyl or C₂-C₈ alkynyl.

Examples of the surfactants contemplated herein for use with dioxetane compounds described herein also include fluorescein surfactants (also referred to herein interchangeably as “fluorescein enhancers”) such as those having the formula V/VI:

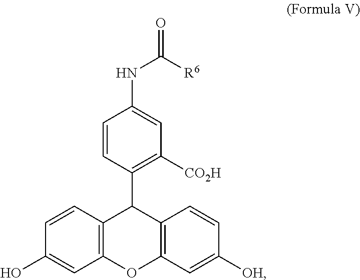

which typically is in equilibrium with:

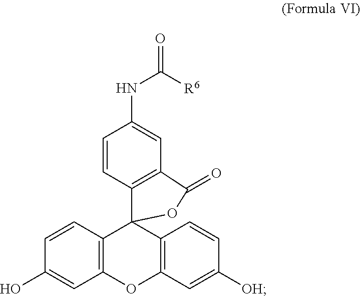

wherein R⁶ is a C₆-C₂₀-alkyl group, such as, for example, C₆-C₁₅-, C₈-C₁₆-, C₁₀-C₁₄-, and C₁₂-C₂₀-alkyl groups. Examples of compounds of the formula V/VI include 5-dodecanoylaminofluorescein, 5-hexadecanoylaminofluorescein, 5-stearylaminofluorescein, and the like. The disclosure also provides an aqueous composition comprising one or more chemiluminescent dioxetane compounds having a peak RLU of greater than 8,000 (for example, at 37° C.); and/or a emission half-life (T_{1/2}) of 3 minutes or less (for example, at 37° C.).

Combinations of any of the aforementioned surfactants/enhancers (for example, combinations of two or more small molecule phosphonium, polymeric phosphonium, and fluorescein surfactants/enhancers) are also contemplated herein. But the use of only one surfactant is also contemplated herein.

The disclosure also provides a method for determining at least one of the presence and concentration an analyte (for example, an ALP or ALP-conjugated to an analyte of interest) in a sample, comprising contacting the sample with a compound of Formula I and monitoring the sample for luminescence.

Advantages, some of which are unexpected, are achieved by various embodiments of the disclosure. For example, at the time the compounds of the Formula I were first developed, the wisdom was that an electron withdrawing group was necessary at the R⁴ position for a bright, chemiluminescent probe. See, for example, ACS Cent. Sci. 2017, 3, 349-358. Based on that wisdom, those of skill in the art would not have expected that compounds of Formula I would be effective, bright, chemiluminescent probes since they lack an electron withdrawing group at the R⁴ position. Instead, the compounds of the Formula I have an electron donating vinyl group at the R⁴ position.

Various compounds and compositions described herein can advantageously provide a rapid, high-intensity luminescent signal in non-aqueous media, aqueous media, or both. It is a significant advantage that assays involving such compounds or compositions can be performed faster than those with compounds lacking the features of the presently described compounds and compositions because a maximum luminescent signal is attained more quickly. Moreover, the compounds and compositions of the disclosure provide increased intensity of luminescence, including in aqueous media. The compounds and compositions of the present disclosure also provide an improved signal to noise ratio and have reduced background signal. Due to such advantageous properties, various embodiments of the disclosure can provide a method or kit that can detect an analyte in an aqueous or non-aqueous sample in less than 3 minutes, less than 1 minute, less than 30 seconds, or less than 15 seconds.

### BRIEF DESCRIPTION OF THE DRAWINGS

FIG. 1 is a plot of relative luminescence units as a function of time (minutes) for the compounds “VPPD,” “VMPD,” and “PPD” using the fluorescein surfactant used in LP530. The fluorescein surfactant used in LP530 comprises a fluorescein surfactant of the formula V/VI and cetyltrimethylammonium bromide (CTAB). “VPPD,” “VMPD,” and “PPD” have the structures further described here.

FIG. 2A is a plot of relative luminescence units as a function of time (minutes) for the compounds “VPPD,” “VMPD,” and “PPD” using “EXL surfactant,” wherein the EXL surfactant has the structure further described herein.

FIG. 2B is a plot of RLU as a function of time at various “VPPD” concentrations (within the range 50 mg/L to 500 mg/L) while keeping EXL concentration constant (within the range 100 mg/L to 250 mg/L).

FIG. 2C is a plot of RLU as a function of time at various “EXL surfactant” concentrations (within the range 100 mg/L to 500 mg/L) while keeping VPPD concentration constant (within the range 50 mg/L to 500 mg/L).

FIG. 3 is a plot of relative luminescence units as a function of time (minutes) for the compounds “VPPD,” “VMPD,” and “PPD” in EXL surfactant, or “VPPD” in the fluorescein surfactant used in LP530, “VMPD” in the fluorescein surfactant used in LP530, or LP530 (that is, PPD in fluorescein surfactant) normalized to the intensity of VPPD in EXL surfactant.

FIG. 4 is a plot of relative luminescence units as a function of time (minutes) for VPPD in various phosphonium surfactants; also shown is relative luminescence units as a function of time (minutes) for LP530.

FIG. 5 is a plot of RLU for “VPPD” in a polymeric phosphonium surfactant as a function of moles of ALP.

FIG. 6 is a plot of RLU as a function of time (minutes) for “VPPD” in “EXL surfactant” and for LP530. In this experiment, a 100 μL solution of VPPD and a polymer phosphonium enhancer (EXL) was mixed with 10 μL of AP10 (1.2×10⁻²¹ mol/μL ALP) and incubated at 37° C.; a 100 μL solution of LP530 was mixed with 10 μL AP8 (1.2×10⁻¹⁹ mol/μL ALP) and incubated at 37° C. Notably, AP8 has a concentration of ALP 100 times greater than that of AP10. That is, 100 times the ALP concentration was needed to get nearly the same light signal with LP530 that is achieved with VPPD and EXL.

FIG. 7 is a plot of RLU as a function of time (minutes) for the VPPD EXL and LP530 backgrounds, where “background” refers to the signal obtained for a VPPD composition comprising EXL or LP530, each without any ALP. The signal is generated from thermal decomposition of the dioxetane when it is incubated at 37° C.

### DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The compounds of the disclosure are useful in chemiluminescent applications, such as assays and chemical probes, for example, for alkaline phosphatase (ALP). One significant benefit of the compositions described herein is that one can improve ALP detection limits. For example, with the compositions described herein, one can detect alkaline phosphatase concentrations from 1×10⁻¹² mol/μL to 1×10⁻²³ mol/μL. For example, in some aspects, the compositions described herein may be used to detect ALP in a composition having a ALP concentration of at least 1×10⁻²³ mol/μL, at least 1×10⁻²² mol/μL, at least 1×10⁻²¹ mol/μL, at least 1×10⁻²⁰ mol/μL, or at least 1×10⁻¹⁷ mol/μL; additionally or alternatively, the compositions described herein may be used to detect ALP in a composition having a ALP concentration of up to 1×10⁻²¹ mol/μL, up to 1×10⁻²⁰ mol/μL, up to 1×10⁻¹⁹ mol/μL, up to 1×10⁻¹⁸ mol/μL, up to 1×10⁻¹⁵ mol/μL, up to 1×10⁻¹⁰ mol/μL, or up to 1×10⁻⁵ mol/μL. In some exemplary aspects, the compositions described herein may be used to detect in AP4, having an ALP concentration of 1.2×10⁻¹⁵ mol/μL; AP6, having an ALP concentration of 1.2×10⁻¹⁷ mol/μL; AP8, having an ALP concentration of 1.2×10⁻¹⁹ mol/μL; and AP9 having an ALP concentration of 1.2×10⁻²⁰ mol/μL. For example, with the compositions described herein, one can detect 1×10⁻¹¹ mol to 1×10⁻²² mol alkaline phosphatase (such as 1×10⁻¹⁴ mol, 1×10⁻¹⁵ mol, 1×10⁻¹⁶ mol, 1×10⁻¹⁷ mol, 1×10⁻¹⁸ mol, 1×10⁻¹⁹ mol, 1×10⁻²⁰ mol, 1×10⁻²¹ mol, 1×10⁻²² mol, 1×10⁻²³ mol; from 1×10⁻¹³ mol to 1×10⁻²² mol, 1×10⁻¹¹ mol to 1×10⁻²⁰ mol, 1×10⁻¹³ mol to 1×10⁻¹⁸ mol, 1×10⁻¹⁵ mol to 1×10²⁰ or 1×10⁻¹⁴ mol to 1×10⁻¹⁷ mol alkaline phosphatase), such as in a composition comprising 5 μL to 50 μL (for example, 10 μL AP9) of an alkaline phosphatase composition diluted in 50 μL to 250 μL (for example, 100 μL) buffer, such as a composition comprising from 1×10⁻¹¹ mol to 1×10⁻²² mol alkaline phosphatase.

In some aspects, the luminescent intensity of the compounds of the disclosure is described in terms of relative light units (RLU). The term “RLU” refers to relative light units in terms of chemiluminescence signal (S) in the presence of an alkaline phosphatase (ALP). In some aspects, RLU may be corrected for background chemiluminescence (B) in the absence of ALP, e.g., S minus B.

Specifically, this disclosure describes compositions that include a combination of a dioxetane and selected surfactants that provide unexpected emission results in aqueous environments. The dioxetane concentration may be selected by a skilled artisan. Exemplary dioxetane concentrations that may be used include 0.5 mg/L to 5000 mg/L (for example, 1 mg/L to 1000 mg/L, 1 mg/L to 500 mg/L, 25 mg/L to 2000 mg/L, 50 mg/L to 500 mg/L, 50 mg/L to 250 mg/L, 100 mg/L to 150 mg/L, 50 mg/L to 1,000 mg/L, 200 mg/L to 1200 mg/L, 50 mg/L to 500 mg/L, 50 mg/L to 150 mg/L, 100 mg/L to 200 mg/L, 100 mg/L to 500 mg/L, and 100 mg/L to 200 mg/L). In some aspects, the dioxetane concentrations that may be used are at least 0.5 mg/L, at least 1 mg/L, at least 25 mg/L, at least 50 mg/L, at least 100 mg/L, at least 200 mg/L, at least 500 mg/L, at least 1000 mg/L, at least 3000 mg/L, or at least 4000 mg/L. In some aspects, the he dioxetane concentrations that may be used may be at most 5000 mg/L, at most 4000 mg/L, at most 3000 mg/L, at most 1000 mg/L, at most 500 mg/L, at most 200 mg/L, 100 mg/L, at most 25 mg/L, or at most 1 mg/L.

The surfactant concentration may also be selected by a skilled artisan. In Exemplary surfactant concentrations that may be used include 5 mg/L to 25,000 mg/L (for example, 1,000 mg/L to 10,000 mg/L, 500 mg/L to 5,000 mg/L, 50 mg/L to 10,000 mg/L, 100 mg/L to 5,000 mg/mL, 100 mg/L to 500 mg/L, 250 mg/L to 1,000 mg/L, 250 mg/L to 500 mg/L, 50 mg/L to 500 mg/L, 50 mg/L to 5,000 mg/L, 500 mg/L to 1,000 mg/L, 100 mg/L to 500 mg/L, 100 mg/L to 1,000 mg/L, 100 mg/L to 300 mg/L, 150 mg/L to 250 mg/L, and 200 mg/L to 300 mg/L). In some aspects, the surfactant concentration that may be used may be at least 5 mg/L, at least 50 mg/L, at least 100 mg/L, at least 500 mg/L, at least 1000 mg/L, at least 3000 mg/L, at least 4000 mg/L, at least 5000 mg/L, at least 10,000 mg/L, at least 15,000 mg/L or at least 20,000 mg/L. In some aspects, the surfactant concentration that may be used may be at most 25,000 mg/L, at most 20,000 mg/L, at most 15,000 mg/L, at most 10,000 mg/L, at most 5000 mg/L, at most 4000 mg/L, at most 3000 mg/L, at most 1000 mg/L, at most 500 mg/L, at most 100 mg/L or at most 50 mg/L.

The ratio of the concentration of dioxetane compound to surfactant may be in a range of 5:1 to 1:10 (for example, 1:1 to 1:5, 1:2 to 1:8; 1:1 to 1:3, 1:2 to 1:5; and 1:3 to 1:9). For example, the ratio of the concentration of dioxetane compound to surfactant can be 5:1, 4:1, 3:1, 2:1, 1:1, 1:2, 1:3, 1:4, 1:5, 1:6, 1:7, 1:8, 1:9, or 1:10.

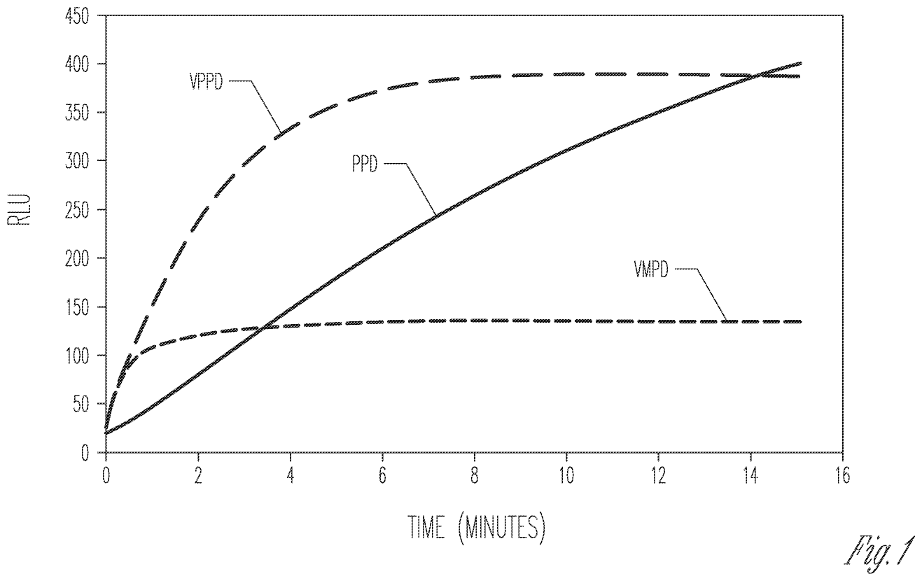

As shown in FIG. 1, a dioxetane compound having the following structure:

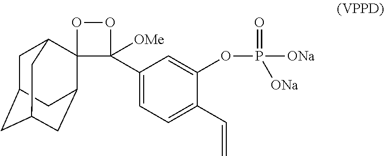

exhibits a higher RLU in a relatively short period of time than a dioxetane compound having the following structure:

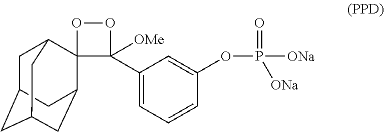

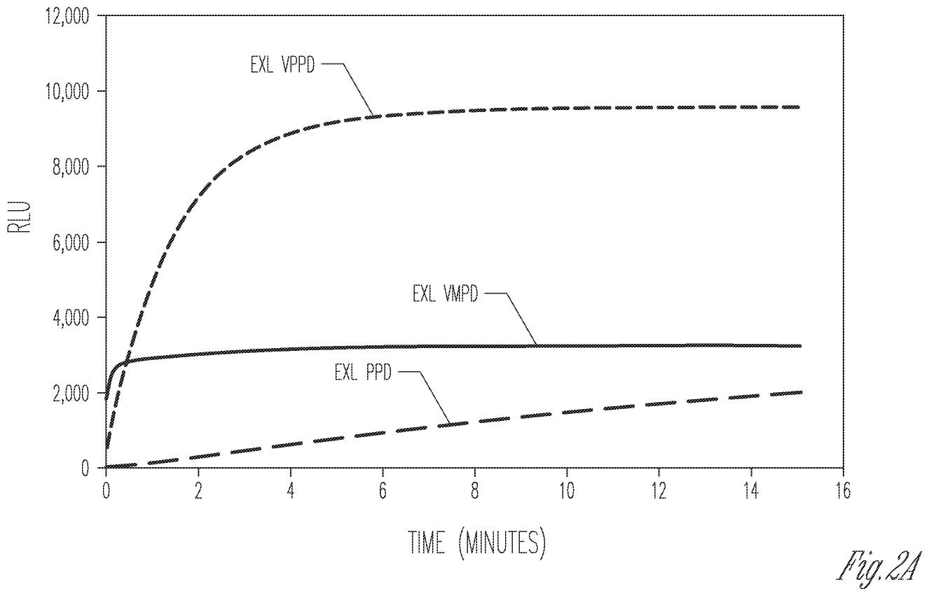

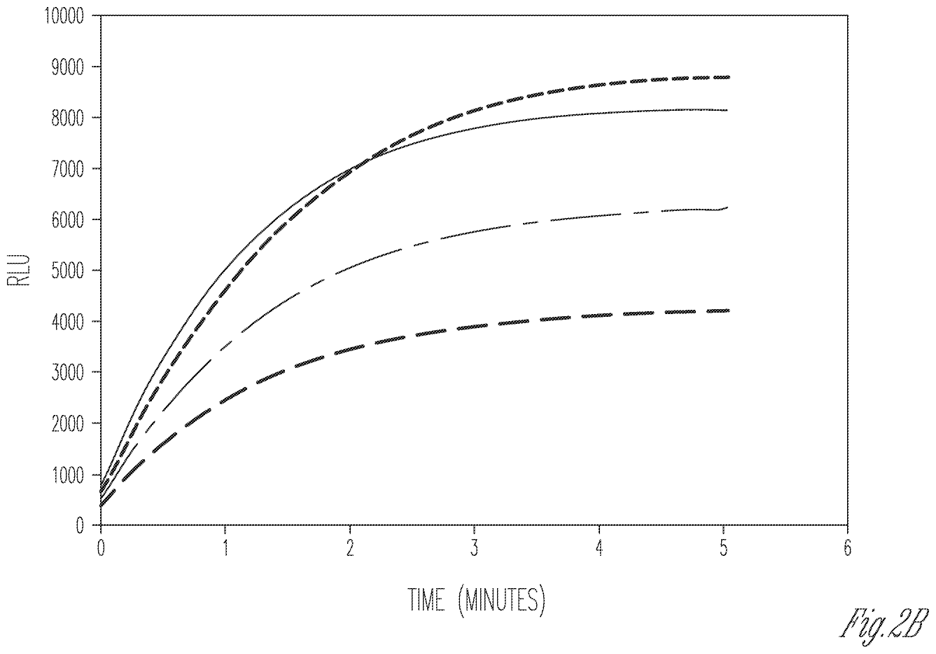

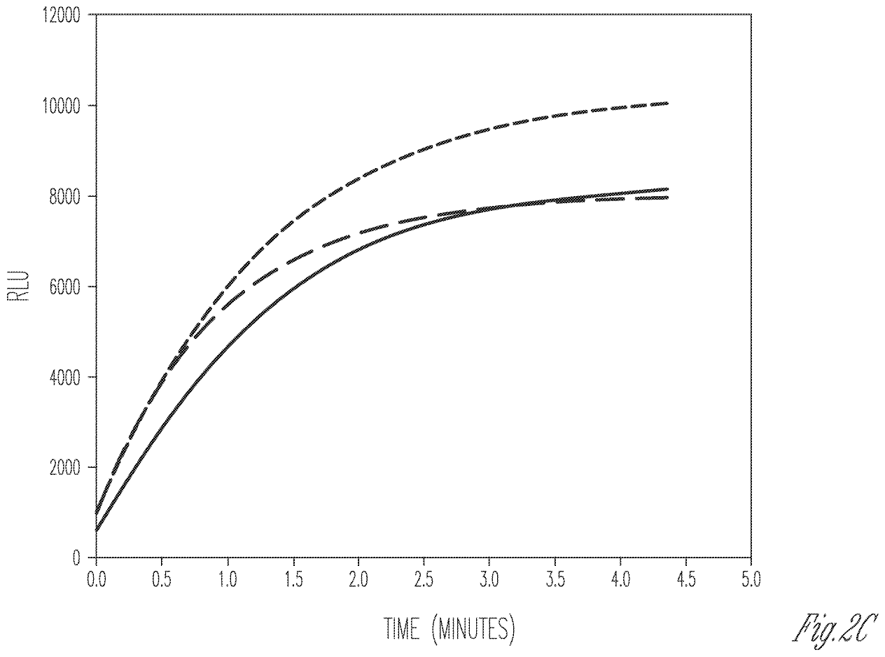

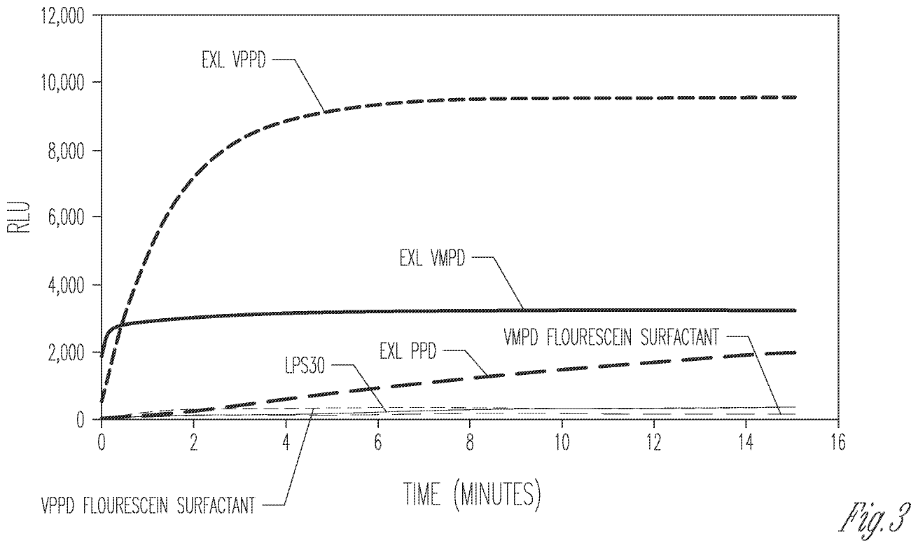

when a fluoresecein surfactant is used (for example, as in LP530). As used herein, the term “LP530” refers to the Lumigen, Inc. product “Lumi-Phos 530,” which comprises the dioxetane compound referenced herein as “PPD” and a fluorescein surfactant. But, as shown in FIGS. 2A and 3, using VPPD in combination with a polymeric phosphonium surfactant/enhancer (EXL) surprisingly results in a dramatic difference in brightness (as shown in relative light units (RLU)) when compared to either PPD or VMPD. For example, VPPD's brightness is over 20 times higher in combination with a polymeric phosphonium surfactant/enhancer relative to VPPD's brightness in combination with a fluoresecein surfactant (compare ~9500 RLU in EXL surfactant vs. ~380 in fluoresecein surfactant). See FIG. 3.

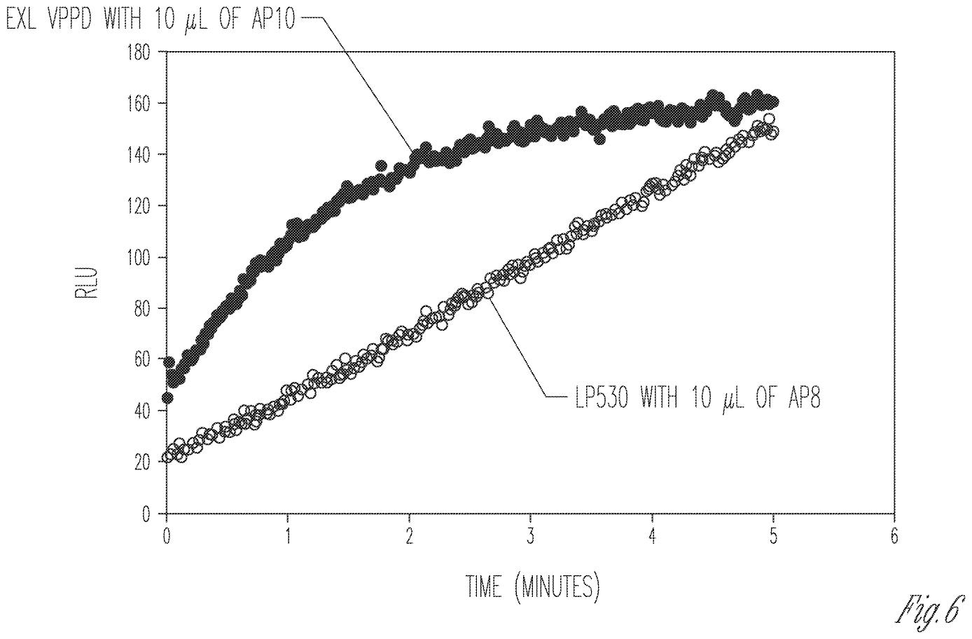

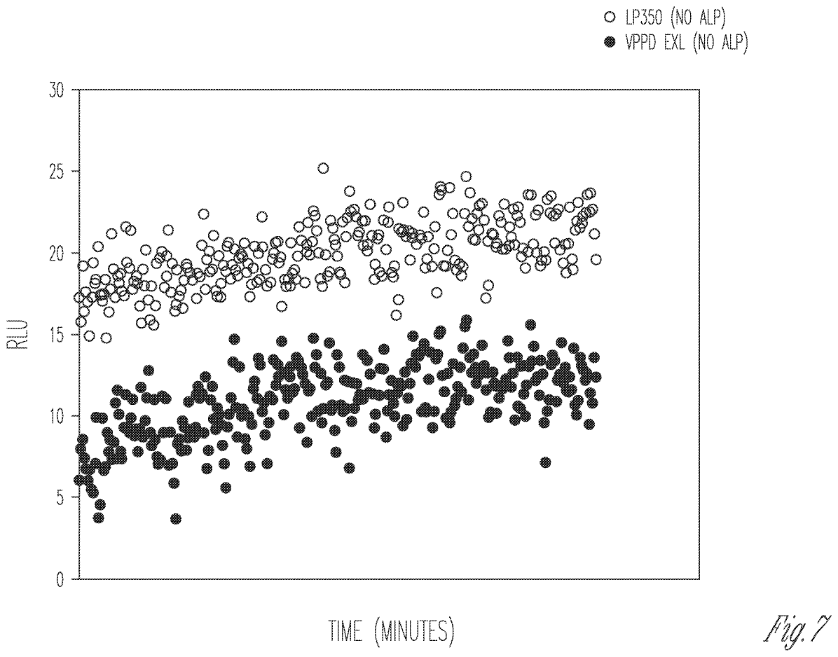

As an added benefit, polymeric phosphonium surfactants/enhancers provide ~50-fold (and as high as a ~100-fold) increase in the signal-to-noise ratio. For example, VPPD in a polymeric phosphonium enhancer can detect AP10 (1.2×10⁻²¹ mol ALP) using 10 μL of AP10 added to a 100 μL solution of VPPD and enhancer. But to generate substantially the same signal with LP530, one needs 100 times more ALP (10 μL AP8; AP8 refers to 1.2×10⁻¹⁹ mol/μL ALP). See FIG. 6. FIG. 7 is a plot of RLU as a function of time for the VPPD EXL and LP530 backgrounds, where “background” refers to the signal obtained for a VPPD composition comprising EXL or LP530, each without any ALP. The signal is generated from thermal decomposition of the dioxetane when it is incubated at 37° C. The background signal for the composition comprising VPPD and EXL is about one third of the signal obtained for LP530. The lower signal without ALP (background), as seen in FIG. 7, means that the limit of detection, that is the lowest actual amount of an analyte conjugated to ALP that can be reliably detected and distinguished from background is also lower.

Moreover, as shown in FIGS. 2A and 3, VPPD's brightness in combination with polymeric phosphonium surfactant/enhancer is greater than other dioxetanes in combination with polymeric phosphonium surfactant/enhancer including, for example, PPD in combination with polymeric phosphonium surfactant/enhancer or VMPD in combination with polymeric phosphonium surfactant/enhancer, where VMPD is a dioxetane compound having the following structure:

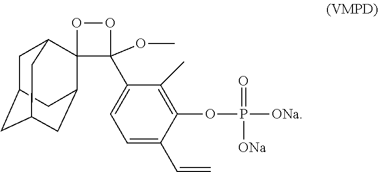

For example, VMPD in combination with polymeric phosphonium surfactant/enhancer has a brightness greater than LP530. Indeed, as shown in these exemplary figures, VMPD in combination with polymeric phosphonium surfactant/enhancers gives a signal that is much brighter than LP530. Further, VMPD in polymeric phosphonium surfactant reaches signal maximum faster than VPPD in the same surfactant (less than 1 minute).

These results suggest that it is the combination of the specific dioxtane compound and the phosphonium surfactant/enhancer that provide unexpectedly improved emission results in aqueous environments.

The disclosure provides a composition comprising a compound of Formula I and salts thereof:

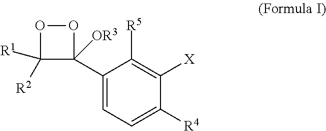

wherein each of R¹ and R² is independently C₃-C₁₀ alkyl, or R¹ and R² taken together with the carbon to which they are attached provide a C₅-C₁₀ cycloalkyl ring;
R³ is C₁-C₁₀ alkyl, C₆-C₁₀ aryl, or heteroaryl;
R⁴ is C₂-C₁₀ alkenyl;
R⁵ is H or C₁-C₁₀ alkyl;
X is a phosphate (for example, a phosphate group of the formula —O—P(O)(ONa)(ONa)); and at least one surfactant.

For example, the disclosure provides a composition comprising a compound of Formula II, or a salt thereof:

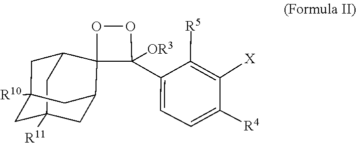

wherein each of R¹⁰ and R¹¹ is independently H, halogen, C₁-C₁₀ alkyl, C₂-C₁₀ alkenyl, C₅-C₁₀ aryl; R³ is C₁-C₁₀ alkyl, C₆-C₁₀ aryl, or heteroaryl; R⁴ is C₂-C₁₀ alkenyl; R⁵ is H or C₁-C₁₀ alkyl; and at least one surfactant. In some embodiments, R¹⁰ and R¹¹ is independently H or halogen.

The disclosure provides a composition comprising a compound of Formula III, or a salt thereof:

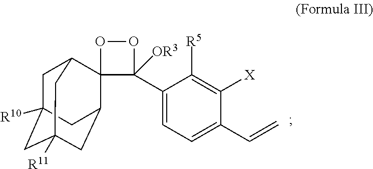

and
at least one surfactant, wherein each of R¹⁰ and R¹¹ is independently H, halogen, C₁-C₁₀ alkyl, C₂-C₁₀ alkenyl, C₆-C₁₀ aryl; R³ is C₁-C₁₀ alkyl, C₆-C₁₀ aryl, or heteroaryl; R⁴ is C₂-C₁₀ alkenyl; R⁵ is H or C₁-C₁₀ alkyl; and at least one surfactant. In some embodiments, R¹⁰ and R¹¹ is independently H or halogen.

The disclosure further provides a composition comprising a compound of Formula IV, or a salt thereof:

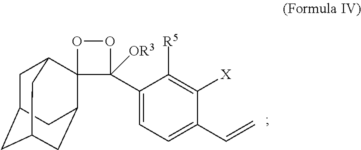

and
at least one surfactant, wherein each of R¹⁰ and R¹¹ is independently H, halogen, C₁-C₁₀ alkyl, C₂-C₁₀ alkenyl, C₆-C₁₀ aryl; R³ is C₁-C₁₀ alkyl, C₆-C₁₀ aryl, or heteroaryl; R⁴ is C₂-C₁₀ alkenyl; R⁵ is H or C₁-C₁₀ alkyl; and at least one surfactant. In some embodiments, R¹⁰ and R¹¹ is independently H or halogen.

The compounds of the formulae I-IV may be a compound of the formula:

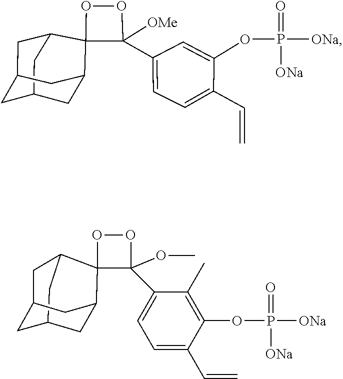

or a salt thereof.

The compositions comprising the compounds of the formulae I-IV may be an aqueous composition or a non-aqueous composition. The composition may be mixture of both aqueous and non-aqueous solvents and may comprise other additives (for example, ions, including magnesium ions, such as magnesium ions derived from magnesium salts such as magnesium chloride, and the like). If the compositions comprising the compounds of the formulae I-IV comprise magnesium ions, they may comprise 50 mg/L to 500 mg/L (for example, 50 mg/L to 250 mg/L, 100 mg/L to 190 mg/L, 150 mg/L to 200 mg/L or 150 mg/L to 190 mg/L) magnesium (II) chloride (MgCl₂), though other magnesium (II) salts can be used. In one example, the magnesium ion concentrations can be from 0 mM to 10 mM, 0 mM to 3 mM, 0.5 mM to 2 mM, 0.5 mM to 1 mM, 1 mM to 5 mM, or 2 mM to 5 mM.

In various embodiments, the surfactants used with compositions comprising the compounds of the formulae I-IV include, but are not limited to, phosphonium surfactants, which are also referred to herein interchangeably as “phosphonium enhancers.” Phosphonium surfactants include polymeric phosphonium surfactants and small molecule phosphonium surfactants. Examples of polymeric phosphonium surfactants include polyvinyl type polymers with pendant quaternary phosphonium groups, which are disclosed in U.S. Pat. No. 5,393,469. For example, U.S. Pat. No. 5,393,469 describes polymeric phosphonium surfactants of the general formulae:

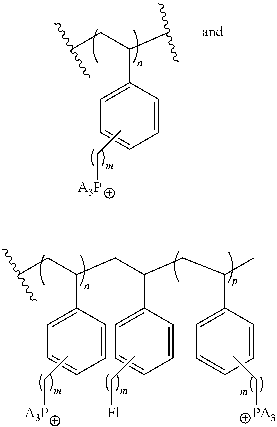

wherein each A is selected from lower alkyl containing 1 to 20 carbon atoms, aryl or aralkyl groups, the group FI is a fluorescent group, m is an integer between 1 and 14, and wherein n and p are integers between about 10 and 1000. Each A groups on a specific phosphorus atom may all be the same group or may be two different groups or all three may be different. The set of A groups on adjacent phosphorus atoms may be the same set or may be different sets wherein the sets are subject to the description above. The relative position of substituents on the aromatic ring may be ortho, meta, para or mixtures of the three types in any proportion. The attached fluorescent group may be any fluorescer which can be chemically linked to a polymer and which has a lower energy for its singlet electronic excited state compared to the excited state of the dioxetane (e.g., compounds of the formulae I-IV). The fluorescent group may enhance the chemiluminescence efficiency of the dioxetane by acting as an energy acceptor which becomes excited and releases the excitation energy in the form of light. Examples of fluorescers useful in practicing the present invention include but is not limited to any fluorescent dye; aromatic compounds including polycyclic aromatic compounds, biphenyls, terphenyls, stilbenes, heteroaromatic and polycyclic heteroaromatic compounds such as acridines, coumarins, phthalocyanines, furans, oxazoles, oxadiazoles, benzothiazoles, quinolines, xanthenes, fluorescein and fluorescein derivatives, for example, amidofluorescein, eosin and eosin derivatives, rhodamines and resorufins.

Exemplary polymeric phosphonium enhancers include polyvinylbenzyltributylphosphonium chloride copolymer with polyvinylbenzyltrioctylphosphonium chloride and polyvinylbenzyltributylphosphonium chloride as well as dicationic compounds bearing two quaternary ammonium or phosphonium groups, such as those disclosed in U.S. Pat. No. 5,451,347. For example, U.S. Pat. No. 5,451,347 describes compounds bearing two quaternary ammonium or phosphonium groups of the formulae 1-27:

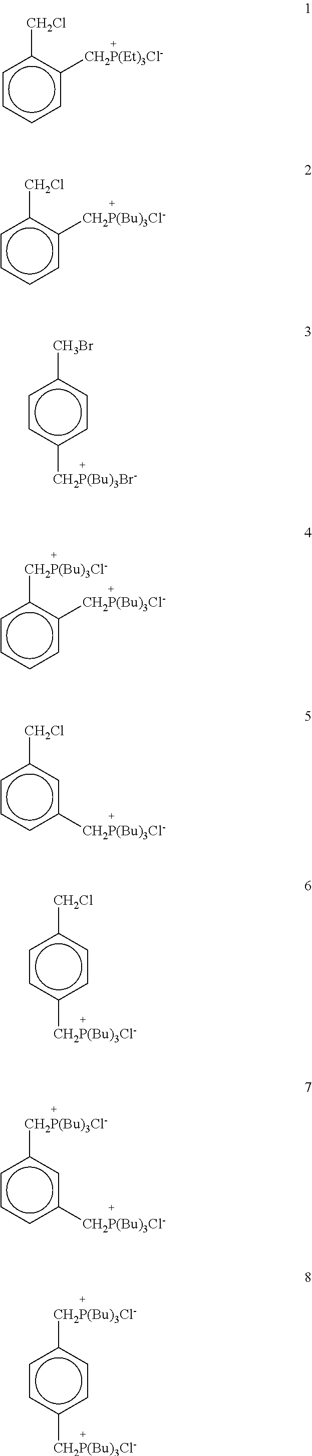

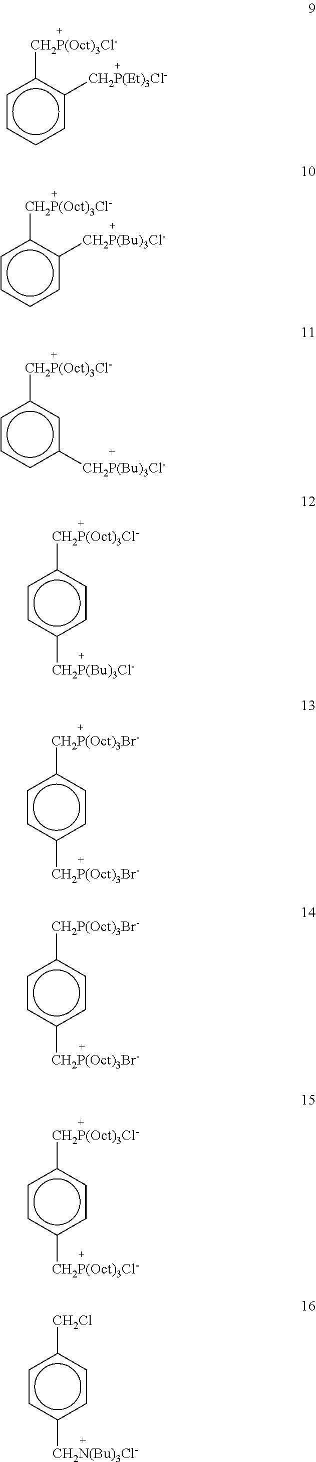

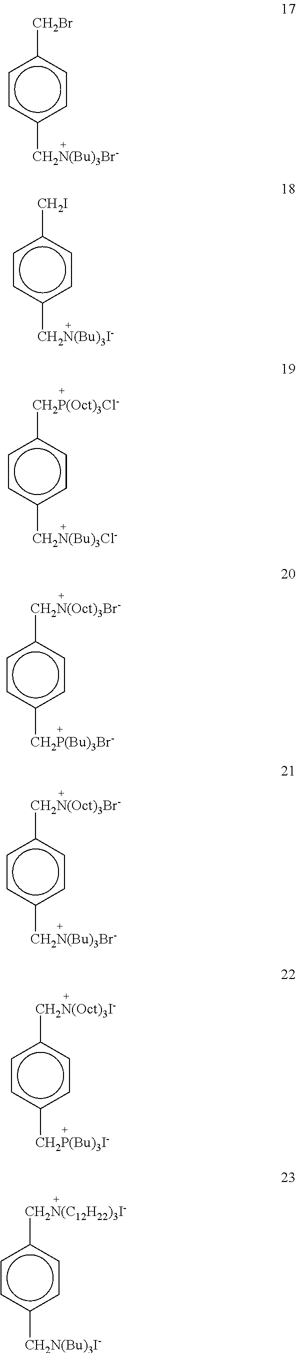

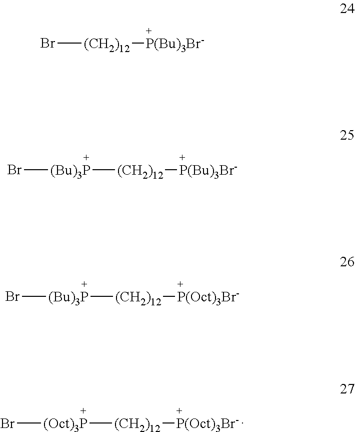

Examples of polymeric phosphonium enhancers include polymers comprising repeating unit (A), repeating unit (B) or both:

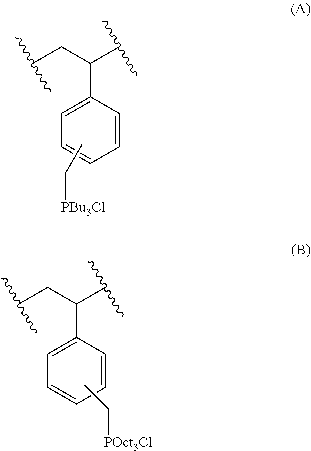

wherein “Bu₃” refers to “tributyl, “Oct₃” refers to “trioctyl.” A molar ratio of the repeating unit (A) to repeating unit (B) may be in a range of from 1:100 to 100:1 (for example, for example, from 100:1 to 11:1, including, for example a ratio of 80:1, 70:1; 60:1, 50:1; 40:1, 30:1, 20:1, 10:1, 9:1, 8:1, 7:1, 6:1, 5:1, 4:1, 70:30, 75:25, 60:40 or 50:50). The weight average molecular weight (M_{w}) may be for example, in a range from 100,000 g/mol to 400,000 g/mol, from 150,000 g/mol to 350,000 g/mol, from 200,000 g/mol to 300,000 g/mol. For example, in exemplary aspects, the M_{w} can be 100,000, 120,000, 140,000, 160,000, 180,000, 200,000, 220,000, 240,000, 260,000, 280,000, 300,000, 320,000, 340,000, 360,000, 380,000, or 400,000 g/mol. In some embodiments, the M_{w} is 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol. The copolymers may be random copolymers, block copolymers, or random-block copolymers.

Examples of polymeric phosphonium enhancers include polymers of the general formula:

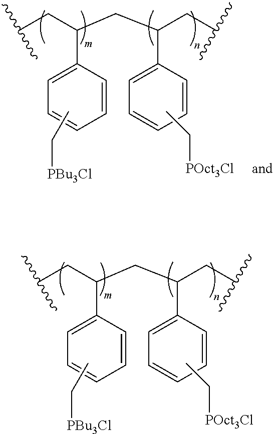

wherein m and n are each, independently, integers from 0 to 1000 (including, for example, integers from 1 to 500, 250 to 1000, 300 to 900, 50 to 500, or 250 to 750). The ratio of m:n may be in a range of from 100:1 to 1:1, 4:1 such as 70:30, 75:25, 60:40 or 50:50; m+n is in range of 100 to 1000 (for example, m+n may be 100 to 600, 200 to 500, or 300 to 600); the weight average molecular weight (M_{w}) may be for example, in a range from 100,000 g/mol to 400,000 g/mol, from 150,000 g/mol to 350,000 g/mol, from 200,000 g/mol to 300,000 g/mol. For example, in exemplary aspects, the M_{w} can be 100,000, 120,000, 140,000, 160,000, 180,000, 200,000, 220,000, 240,000, 260,000, 280,000, 300,000, 320,000, 340,000, 360,000, 380,000, or 400,000 g/mol. In some embodiments, the M_{w} is 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol. The copolymers may be random copolymers, block copolymers, or random-block copolymers.

Exemplary polymeric phosphonium enhancers/surfactants also include polymeric phosphonium surfactants comprising the structure:

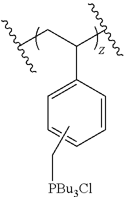

wherein z is an integer in a range from 2 to 1000 (for example, 2 to 500, 100 to 600, 450 to 800, 300 to 600, 2 to 250, 50 to 250, 100 to 300, 150 to 400, 175 to 400, 50 to 300, 100 to 500, 150 to 400, or 10 to 200). The weight average molecular weight (M_{w}) may be for example, in a range from 500 g/mol to 500,000 g/mol, from 500 g/mol to 100,000 g/mol, 50,000 g/mol to 100,000 g/mol, from 150,000 g/mol to 350,000 g/mol or from 200,000 g/mol to 300,000 g/mol. For example, in exemplary aspects, the M_{w} can be 50,000, 60,000, 70,000, 100,000, 120,000, 140,000, 160,000, 180,000, 200,000, 220,000, 240,000, 260,000, 280,000, 300,000, 320,000, 340,000, or 350,000 g/mol. In some embodiments, the M_{w} is 70,000, 80,000, 100,000 or 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol. Examples of such polymeric phosphonium enhancers/surfactants include those comprising the structures:

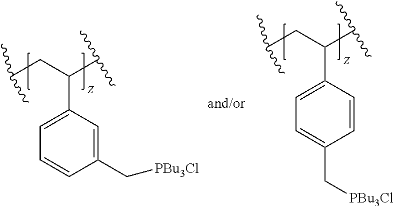

wherein z is an integer from 2 to 1000 (for example, 2 to 500, 100 to 600, 450 to 800, 300 to 600, 2 to 250, 50 to 250, 100 to 300, 150 to 400, 175 to 400, 50 to 300, 100 to 500, 150 to 400, or 10 to 200). The weight average molecular weight (M_{w}) may be for example, in a range from 500 g/mol to 500,000 g/mol, from 500 g/mol to 100,000 g/mol, 50,000 g/mol to 100,000 g/mol, from 150,000 g/mol to 350,000 g/mol or from 200,000 g/mol to 300,000 g/mol. For example, in exemplary aspects, the M_{w} can be 50,000, 60,000, 70,000, 100,000, 120,000, 140,000, 160,000, 180,000, 200,000, 220,000, 240,000, 260,000, 280,000, 300,000, 320,000, 340,000, or 350,000 g/mol. In some embodiments, the M_{w} is 70,000, 80,000, 100,000 or 220,000 g/mol. In some embodiments, the M_{w} is 240,000 g/mol. In some embodiments, the M_{w} is 260,000 g/mol. In some embodiments, the M_{w} is 280,000 g/mol. In some embodiments, the M_{w} is 300,000 g/mol.

Exemplary small molecule phosphonium enhancers/surfactants include small molecule surfactants of the formula:

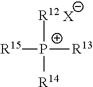

wherein:
R¹²-R¹⁴ are each, independently, C₁-C₁₀ alkyl;
R¹⁵ is arylalkyl; and
X⁻ is a counterion (for example, chloride). R¹⁵ may be arylalkyl substituted with a C₂-C₈ alkenyl group or with a group of the formula:

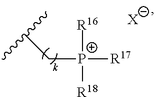

wherein k is 0, 1, or 2; R¹⁶-R¹⁸ are each, independently, C₁-C₁₆ alkyl. R¹⁶-R¹⁸ may each, independently, be C₄-C₁₀ alkyl. The C₂-C₈ alkenyl group may be a group of the formula:

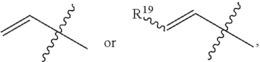

wherein R¹⁹ is C₁-C₈ alkyl, C₂-C₈ alkenyl or C₂-C₈ alkynyl.

Examples of small molecule phosphonium enhancers/surfactants include:

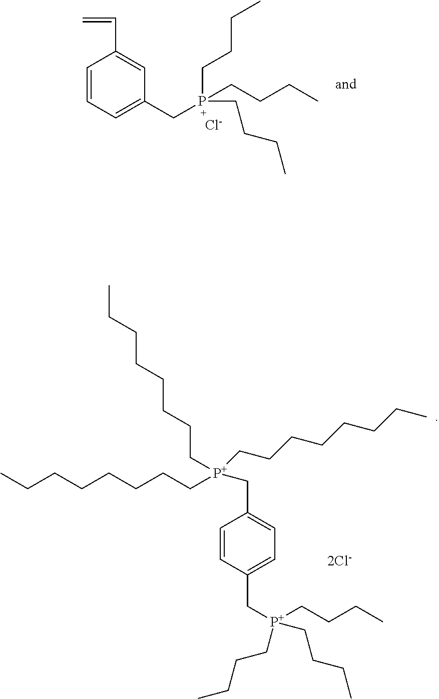

Other surfactants contemplated herein include poly(vinylbenzyl tributylphosphonium chloride) (TBE) (for example, TBE having a molecular weight of 175,000 g/mol), polyvinylpyridinium salts (for example, polyvinylpyridinium salts having molecular weights from 10,000 g/mol to 100,000 g/mol), alkyl ammonium salts (for example, (C₃-C₂₀-alkyl)₂N(C₁-C₄-alkyl)₂Cl, such as (C₃H₁₇)₃N(CH₃)Cl, (C₁₂H₂₅)₂N(CH₃)₂Cl and (C₁₈H₃₇)₂N(CH₃)₂), alkyl-glycol ammonium salts (for example, (C₁₂-C₂₀-alkyl)₂N(C₁-C₄-alkyl-O)₂Cl, such as (C₁₂H₂₅)₂N((CH₂CH₂O)₅)₂Cl and (C₁₈H₃₇)₂N((CH₂CH₂O)₅)₂Cl), and arylalkyl-alkyl ammonium salts (for example, (C₁₈H₃₇)₂N(CH₃)(CH₂Ph)Cl).

Combinations of any of the aforementioned surfactants/enhancers (for example, combinations of two or more of small molecule phosphonium, polymeric phosphonium, and fluorescein surfactants/enhancers) are also contemplated herein. For example, a composition can comprise a small molecule phosphonium and a polymeric phosphonium. In some instances, a composition comprises a small molecule phosphonium and a fluorescein surfactant/enhancer. In some instances, a composition comprises a small molecule phosphonium, a polymeric phosphonium, and a fluorescein surfactant/enhancer. For example, a composition can comprise a combination as follows:
(a) of a polymeric phosphonium of the formula:

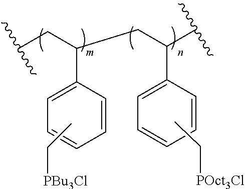

and a small molecule phosphonium of the formula

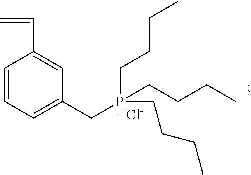

(b) a polymeric phosphonium of the formula:

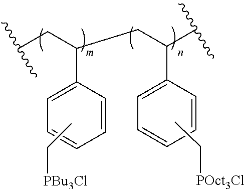

and a small molecule phosphonium of the formula

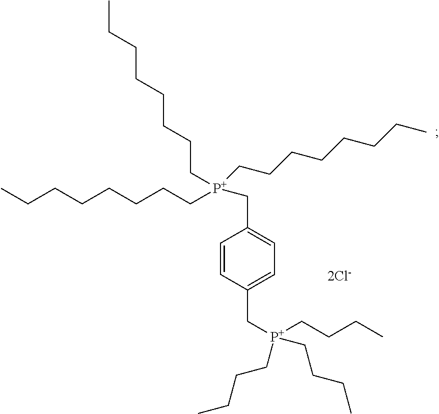 [SMILES: CCCCCCCC[P+](CCCCCCCC)(CCCCCCCC)Cc1ccc(C[P+](CCCC)(CCCC)CCCC)cc1]

(c) a small molecule phosphonium of the formula

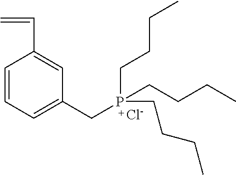

and
a fluorescein surfactant/enhancer of the formula:

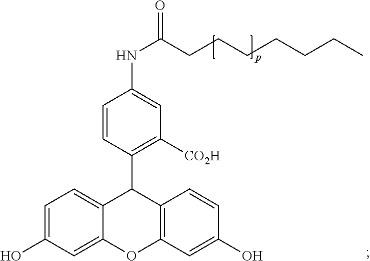

(d) a small molecule phosphonium of the formula

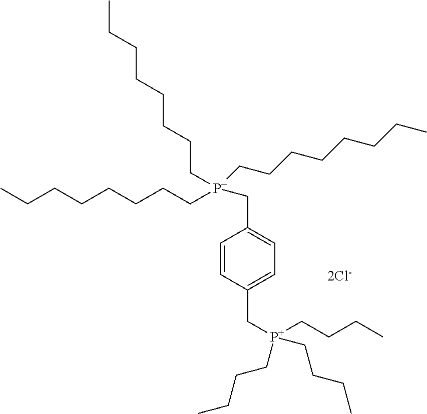 [SMILES: CCCCCCCC[P+](CCCCCCCC)(CCCCCCCC)Cc1ccc(C[P+](CCCC)(CCCC)CCCC)cc1]

and
a fluorescein surfactant/enhancer of the formula:

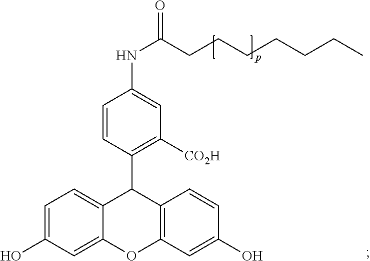

(e) a polymeric phosphonium of the formula:

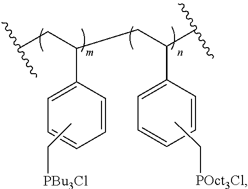

a small molecule phosphonium of the formula:

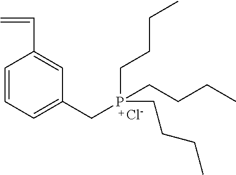

and
a fluorescein surfactant/enhancer of the formula:

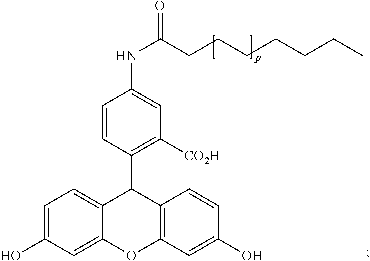

or
(f) a polymeric phosphonium of the formula:

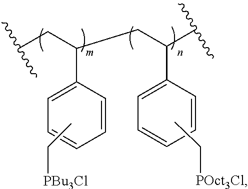

a small molecule phosphonium of the formula:

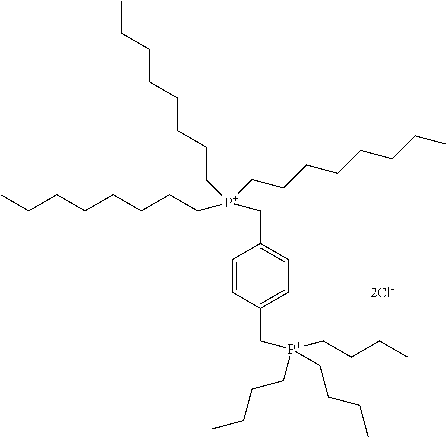 [SMILES: CCCCCCCC[P+](CCCCCCCC)(CCCCCCCC)Cc1ccc(C[P+](CCCC)(CCCC)CCCC)cc1]

and
a fluorescein surfactant/enhancer of the formula:

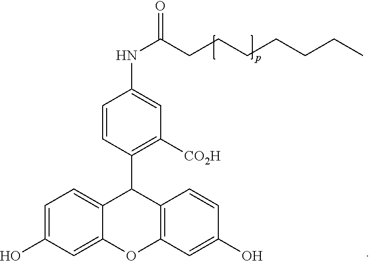

In various embodiments, the composition contains a buffer solution. The buffer solution can, but need not necessarily be, an alkaline or amine-based buffer solution. An example amine-based buffer is 221 buffer (2-amino-2-methyl-1-propanol (AMP) based buffer) available from Sigma-Aldrich (St. Louis, MO). Accordingly, buffer solutions contemplated herein include amine-based buffer solutions comprising 2-amino-2-methyl-1-propanol (AMP), 2-amino-2-methyl-1,3-propanediol (AMPD), Tris (2-amino-2-(hydroxymethyl)propane-1,3-diol), TAPS (3-{[1,3-dihydroxy-2-(hydroxymethyl)propan-2-yl]amino}propane-1-sulfonic acid), Bicine (N,N-bis(2-hydroxyethyl)glycine), and Tricine (N-[1,3-Dihydroxy-2-(hydroxymethyl)propan-2-yl]glycine) and combinations thereof, such as a buffer solution comprising AMPD and AMP. Likewise, the composition can, but need not necessarily have, a basic pH. In some exemplary embodiments, the composition can have a pH of 4 to 12, 5 to 12, 6 to 12, 8 to 11, 7 to 12, 8 to 12, 9 to 12, 10 to 12, 4 to 11, 4 to 10, 4 to 9, 4 to 8, 4 to 7, 4 to 6, or 4 to 5, for example, at a buffer concentration of 0.1 to 1 M, 0.1 to 0.5 M, 0.2 to 0.5 M, 0.2 to 0.8 M, 0.1 to 0.25 M, 0.2 to 0.3 M, or 0.15 M to 0.3M. The pH of the composition can be selected based on whether luminescence is intended to trigger immediately upon analyte-triggered removal of the X-group of the compounds described herein by ALP, as is typically the case for alkaline pH values, or the pH of the composition can be acidic so as to luminesce upon treatment with base.

In various embodiments, the composition has/exhibits a RLU of greater than 2,000 RLU, greater than 4,000 RLU, greater than 6,000 RLU, greater than 8,000 RLU, greater than 9,000 RLU; up to 50,000 RLU, up to 30,000 RLU, up to 25,000 RLU, up to 20,000 RLU, up to 15,000 RLU, up to 10,000 RLU, or up to 5000 RLU; from 2,000 RLU to 10,000 RLU, from 4,000 RLU to 10,000 RLU, from 2,000 RLU to 5,000 RLU, from 2,500 RLU to 4,000 RLU or from 8,000 RLU to 10,000 RLU in the presence of ALP or ALP-conjugated to an analyte of interest. RLU may be measured by any suitable means including, for example, using Tuner TD-20 luminometer to measure luminosity at 37° C. In some aspects, 10 μL of an ALP solution (for example, AP8) may be mixed with 100 μL of formulated reagents. In addition, or alternatively, the composition exhibits a T_{1/2} of 4 minutes or less, 3 minutes or less, 2 minutes or less, 1 minute or less, from 30 seconds to 4 minutes, 30 seconds to 2 minutes, 30 seconds to 1 minute, 1 minute to 3 minutes, or 1 minute to 2 minutes (for example at 37° C.).

In various embodiments, the composition has/exhibits a peak RLU in the absence of enzyme (e.g., ALP) of at most 20, at most 19, at most 18, at most 17, at most 16, at most 15, at most 14, at most 13, at most 12 or at most 10. In various embodiments, the composition has/exhibits a peak RLU in the absence of enzyme (e.g., ALP) of at least 0.5, at least 1, at least 2, at least 3, at least 4, at least 5, at least 6, or at least 7. In various embodiments, the composition has/exhibits a peak RLU in the absence of enzyme (e.g., ALP) in a range of 1 to 20; 1 to 15; 1 to 14; 1 to 13; 1 to 12; 2 to 20; 2 to 15; 2 to 14; 2 to 13; 2 to 12; 3 to 20; 3 to 15; 3 to 14; 3 to 13; or 3 to 12.

The compositions comprising the dioxetane and surfactant described herein may contain magnesium salts in an amount of from 50 mg/L to 500 mg/L (for example, 50 mg/L to 250 mg/L, 100 mg/L to 190 mg/L, 150 mg/L to 200 mg/L or 150 mg/L to 190 mg/L) magnesium (II) chloride (MgCl₂), though other magnesium (II) salts can be used.

The disclosure also provides a method of detecting an analyte (for example, an ALP or ALP-conjugated to an analyte of interest) in a sample, comprising contacting the sample with one or more of the compounds described herein, or a salt thereof, or a composition comprising the same, and then monitoring the sample for luminescence. In various embodiments, the method involves measuring the intensity of a resulting luminescence and correlating the intensity to at least one of the presence and concentration the analyte (for example, an ALP or ALP-conjugated to an analyte of interest). In various embodiments, the method detects an ALP concentration of at most 1×10⁻¹⁸, 1×10⁻¹⁹, 1×10⁻²⁰, or 1×10⁻²¹ mol/μL. In various embodiments, the method detects an ALP concentration of at least 1×10⁻²² mol/μL or at least 1×10⁻²³ mol/μL. In various embodiments, the method detects an ALP concentration in a range of 1×10⁻²³ mol/μL to 1×10⁻¹⁸ mol/μL (e.g., 1×10⁻²³ mol/μL to 1×10⁻¹⁹ mol/μL; 1×10⁻²² mol/μL to 1×10⁻¹⁸ mol/μL; or 1×10⁻²³ mol/μL to 1×10⁻²⁰ mol/μL). In various embodiments, the method detects at least 1×10⁻²² mol ALP or at least 1×10⁻²¹ mol ALP. In various embodiments, the method detects up to 1×10⁻¹⁷, up to 1×10⁻¹⁸, up to 1×10⁻¹⁹, or up to 1×10⁻²⁰ mol ALP. In various embodiments, the method detects ALP in a range of 1×10⁻²² mol ALP to 1×10⁻¹⁷ mol ALP (e.g., 1×10⁻²² mol ALP to 1×10⁻¹⁸ mol ALP; 1×10⁻²² mol ALP to 1×10⁻¹⁹ mol ALP; or 1×10⁻²¹ mol ALP to 1×10⁻¹⁷ mol ALP).

In some embodiments, the method further involves increasing the pH of the sample. For example, the pH can be adjusted to 7 or higher, 8 or higher, 9 or higher, 10 or higher or 11 or higher.

In various embodiments, the analyte is detected in 10 minutes or less, 6 minutes or less, 4 minutes or less, 2 minutes or less, 1 minute or less, 55 seconds or less, 45 seconds or less, or 30 seconds or less.

The disclosure further provides a kit for determining at least one of the presence and concentration an analyte. In some embodiments, the kit comprising the compound of any one or more of the compounds described herein, an olefin precursor thereof, a salt thereof, or a composition comprising the same. The kit can contain instructions according to the method described herein.

The compounds and compositions described herein can be triggered directly by the addition of an analyte so as to produce a signal identifying at least one of the presence and concentration the analyte and probe or can be triggered in a two step-process, one step which involves contacting the analyte and another step which involves raising the pH.

The compounds of the disclosure can be configured as probes to detect ALP or ALP-conjugated to an analyte of interest.

It is desirable that the compounds emit light in the briefest possible period of time upon being triggered by the analyte so as to provide the strongest signal possible. When the chemiluminescence is emitted gradually over a period of time, light intensity (photons/sec) is diminished and detection sensitivity can be impaired.

The rate of luminescence increase, or rise time, can be described according to either the time to the maximum emission (tₘₐₓ) or the emission half-life (T_{1/2}).

The compounds described herein can be used as an enzyme substrate for alkaline phosphatase (ALP) enzyme, and the like. Without being limited by theory, one example mechanism involves the ALP enzyme hydrolyzing X, wherein X is a phosphate group, to provide a phenol which is immediately deprotonated due to the alkaline environment of the solution (for example, pH 9.7 buffer). Formation of an oxy anion triggers decomposition of the 1,2-dioxetane into two compounds: 2-adamantanone and a phenyl ester that is at an excited state. The excited phenyl ester then immediately decays to the ground state by releasing light.

The resulting light intensity is a linear function of the amount of the enzyme. The compounds described herein can thus be used to detect a label enzyme used in an assay. For example, the steps of the chemical process in which the dioxetane provides light can be described according to the following steps: (i) X+S→X+S′ (ii) S′→P* and (iii) P*→P+ light. Step (i) represents catalytic turnover of the substrate, wherein X is an enzyme or other component that converts the substrate (S) to its activated form (S′), step (ii) represents degradation of the activated substrate to a transient excited species (P*), and step (iii) represents decay of the excited species to ground state (P) and emission of light. Light intensity is the product of the catalytic turnover of substrate in step (i) and the lifetime of the resulting light-producing compound P* in step (ii). Step (ii) is usually first order with a rate constant k and can be characterized by its half-life: T_{1/2}=(ln 2)/k. Step (iii) is extremely short in comparison to the other steps and generally has no meaningful effect on reaction kinetics.

Chemiluminescence intensity/time profile comprises a period of initial rising emission intensity and a subsequent period of steady-state intensity. A slow first order reaction of S′→P* corresponds to an extended rise time as it takes longer for the steady state concentration of S′ to be reached. Fast reaction of S′→P* corresponds to shorter initial rising period and thus provides a rapid rise. For enzymatic chemiluminescent reactions, intensity will typically remain plateaued at a high level. The absence of a steady intensity indicates either substrate depletion or subsequent inactivation of the enzyme. Detection of enzyme-generated chemiluminescence provides flexibility in the measurement process as light intensity at any time point can be related to the amount of enzyme, however enzyme generated processes can have disadvantages, for example, due to the size and “sticky” nature of the enzyme label. However, for maximal sensitivity measurement is optimally conducted at or near the maximum intensity (Iₘₐₓ) during the period of steady-state intensity.

Compounds of the disclosure can generally be prepared, for example, according to the synthetic approach described in Scheme 1.

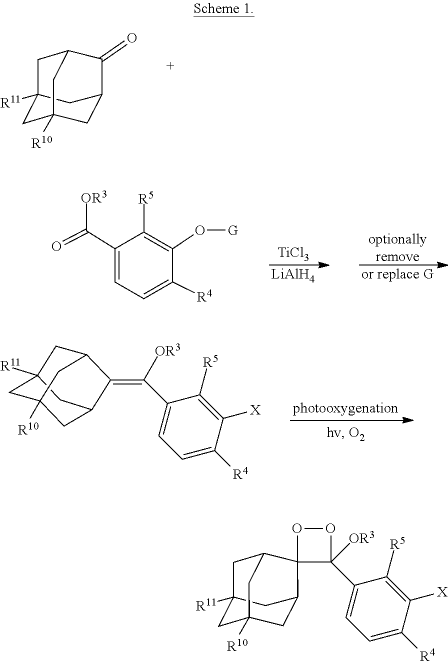

Wherein G is any suitable oxygen protecting group.

Briefly, 2-adamantanone and a 3-substituted benzoate ester can be coupled together by subjecting them to McMurry reaction conditions involving oxophilic titanium and a reducing agent. The resulting olefin can be further modified, for example, via removing or replacing protecting group G or further functionalizing position R⁴. Next, the olefin is subjected to photooxygenation conditions to provide a 1,2-dioxetane product. In various embodiments, R⁴ and X are as described in any of the various embodiments of this application. In some embodiments, R¹⁰ and R¹¹ are H. In some embodiments, R³ is substituted or unsubstituted alkyl.

The term “intensity” (for example, light intensity or luminescence intensity) as used herein refers to the rate of emission in photons/sec. Intensity can be measured by use of a luminometer. A luminometer is a photodetector in a housing which excludes ambient light. Any suitable luminometer can be used, including photomultiplier tubes and/or photodiodes.

The term “speed of luminescence” refers to the rate of luminescence increase, that is, the change in light intensity over time.

The term “sensitivity” as used herein refers to the lowest level at which a signal for an analyte or product being measured can be reproducibly detected.

The term “alkyl” as used herein refers to substituted or unsubstituted straight chain, branched or cyclic, saturated mono- or bi-valent groups having from 1 to 20 carbon atoms, 10 to 20 carbon atoms, 12 to 18 carbon atoms, 6 to 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)-alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, bicyclo[2.2.1]heptyl, and adamantyl. Cycloalkyl groups further include substituted and unsubstituted polycyclic cycloalkyl groups such as, but not limited to, norbornyl, adamantyl, bornyl, camphenyl, isocamphenyl, and carenyl groups, and fused rings such as, but not limited to, decalinyl, and the like. For example, cycloalkyl includes an adamantyl substituted by one, two, three, four, or more substituents, for example, at the tertiary bridgehead positions at the methylene bridges. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl.

The term “alkenyl” as used herein refers to substituted or unsubstituted straight chain, branched or cyclic, saturated mono- or bi-valent groups having at least one carbon-carbon double bond and from 2 to 20 carbon atoms, 10 to 20 carbon atoms, 12 to 18 carbon atoms, 6 to 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, for example, vinyl, propen-1-yl and buten-1-yl, or the alkenyl group can be attached via a portion of the alkenyl group that does not contain the double bond, for example, penten-4-yl. Where specified, the parent moiety should be understood to be attached to the alkenyl group at a vinylic position of the double bond rather than a non-vinylic position. For example, where an aromatic ring is substituted with a r-conjugated alkenyl group, it should be understood to be substituted at the vinyl position rather than a non-vinylic position. As a further example, an aromatic ring substituted with a r-conjugated propenyl group would be understood to be a propen-1-yl or a propen-2-yl group rather than a propen-3-yl group. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)-alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments, alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. The term “heteroaryl” is a fully aromatic heterocyclyl and thus a subset of the term heterocyclyl. The term “heterocycloalkenyl” refers to a heterocyclyl group containing an olefin within a non-aromatic ring, such that the olefin is the point of connection to the parent moiety. A heterocyclyl group can thus be a heterocycloalkyl, heterocycloalkenyl, or a heteroaryl, or if polycyclic, any combination thereof. In some embodiments, heterocyclyl groups include 3 to 20 ring members, whereas other such groups have 3 to 15 ring members. In some embodiments, heterocyclyl groups include heterocyclyl groups that include 3 to 8 carbon atoms (C₃-C₈), 3 to 6 carbon atoms (C₃-C₈) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase heterocyclyl group includes fused ring species including those that include fused aromatic and non-aromatic groups. Representative heterocyclyl groups include, but are not limited to piperidynyl, pyrrolidinyl, piperazinyl, and morpholinyl. For example, heterocyclyl groups include, without limitation:

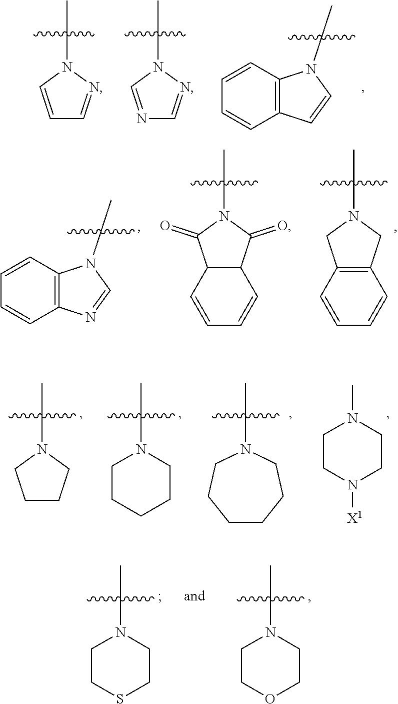

wherein X¹ represents H, (C₁-C₂₀)alkyl, (C₆-C₂₀)aryl or an amine protecting group (for example, a t-butyloxycarbonyl group) and wherein the heterocyclyl group can be substituted or unsubstituted. Representative heteroaryl groups include furanyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. In some embodiments, the heteroaryl is a 5-membered heteroaryl. In some embodiments, the heteroaryl is other than pyridine, pyrimidine, pyridazine, pyrazine, or fused derivatives thereof. A r-excessive heteroaryl is a heteroaryl that is electron-rich such that it can function as an electron donating group. Examples of r-excessive heteroaryls are furan, thiophene, indole, pyrrole, benzofuran, and benzothiophene.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to 12-20 or 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein. The point of substitution to the parent moiety is at the oxygen atom.

The term “arylcarbonyl” as used herein refers to a carbonyl (CO) group connected to an aryl group as are defined herein. The point of substitution to the parent moiety is at the carbonyl group.

The term “heteroarylcarbonyl” as used herein refers to a carbonyl (CO) group connected to an heteroaryl group as are defined herein. The point of substitution to the parent moiety is at the carbonyl group.

The term and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkenyl group is replaced with a bond to an aryl group as defined herein. The point of substitution to the parent moiety is at the alkyl group.

The terms “halo,” “halogen,” or “halide” group, as used herein, by themselves or as part of another substituent, mean, unless otherwise stated, a fluorine, chlorine, bromine, or iodine atom.

The term “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, alkoxy, aryl, cycloalkyl, heterocyclyl, group or the like.

The term “formyl” as used herein refers to a group containing an aldehyde moiety. The point of substitution to the parent moiety is at the carbonyl group.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to a nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” as used herein refers to a sulfur atom connected to an alkyl, alkenyl, or alkynyl group as defined herein. The point of substitution to the parent moiety is at the sulfur atom.

The term “arylthio” as used herein refers to a sulfur atom connected to an aryl group as defined herein. The point of substitution to the parent moiety is at the sulfur atom.

The term “alkylsulfonyl” as used herein refers to a sulfonyl group connected to an alkyl, alkenyl, or alkynyl group as defined herein. The point of substitution to the parent moiety is at the sulfonyl group.

The term “alkylsulfinyl” as used herein refers to a sulfinyl group connected to an alkyl, alkenyl, or alkynyl group as defined herein. The point of substitution to the parent moiety is at the sulfinyl group.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups. The point of substitution to the parent moiety is at the sulfonyl group.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups. The point of substitution to the parent moiety is at the nitrogen atom.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups. The point of substitution to the parent moiety is at the amido group.

Each of the various substituent groups described herein can be substituted or unsubstituted. The term “substituted” as used herein (for example, in the context of “optionally substituted arylalkyl”) refers to a group that is substituted with one or more groups (substituents) including, but not limited to, the following groups: deuterium (D), halogen (for example, F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, for example, an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups. In various embodiments, a substituted group may be substituted with a group other than a carbonyl-containing group, nitro, cyano, sulfinyl, sulfonyl, or a halogen-containing group. In various embodiments, a substituted group may be substituted with a group other than an electron-withdrawing group. Some substituted groups in certain embodiments may be substituted solely with one or more electron-donating groups.

As used herein, the term alcohol protecting group refers to a substituent group on an oxy group which renders the oxygen inert to various conditions in which an alcohol would typically react, but which is readily removed when subjected to certain conditions. Alcohol protecting groups as described herein will typically improve the stability of the dioxetane moiety, and upon their removal will promote decomposition of the dioxetane. Thus, alcohol protecting groups include phosphates such as PO₃Na₂, PO₃Cl₂, and PO₃H₂, glycosyl groups, dinitrobenzenesulfonylaminobenzyl groups, and other groups which can be enzymatically hydrolyzed to provide the unprotected alcohol. Some alcohol protecting groups are described in Theodora W. Greene, Peter G. M. Wuts (1999). Protecting Groups in Organic Synthesis (3 ed.). J. Wiley. Alcohol protecting groups include acetyl, benzoyl, benzyl, methoxyethoxymethyl, dimethyltrityl, methoxylmethyl, methylthiomethyl, pivaloyl, tetrahydropyranyl, tetrahydrofuranyl, trityl, trialkylsilyl, trialkylsiloxymethyl, dialkylarylsilyl, glycosyl, pyranyl, galactosyl, and ethoxyethyl groups. Alcohol protecting groups also include groups in which the alcohol is substituted with a fragmentable linker that is further substituted with a protecting group, wherein upon deprotecting of such protecting group the linker fragments and eliminates from the alcohol. The following compounds are yet further examples of alcohols substituted with an alcohol protecting group:

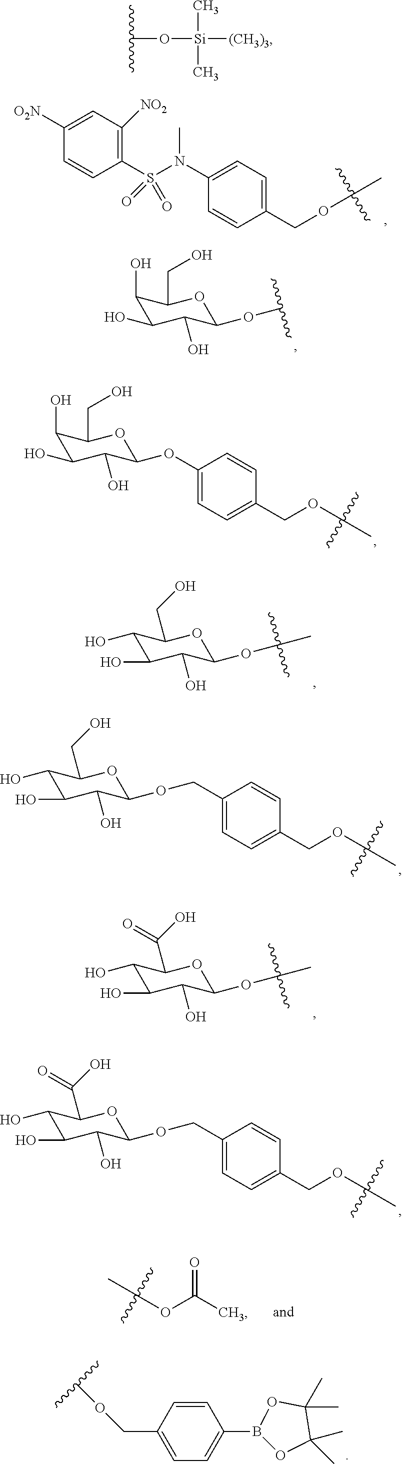

In various embodiments, the protecting group G may be an enzyme-cleavable group, wherein removal of said cleavable group by the analyte of interest, for example, in the presence of an enzyme capable of cleaving said enzyme cleavable group, provides the unstable phenolate-dioxetane species that subsequently decomposes and emits light. For example, G may be a peptide moiety consisting of two or more amino acid residues cleavable by a specific enzyme.

In some instances, the compounds described herein (for example, the compounds of the Formulae I-IV) can contain chiral centers. All diastereomers of the compounds described herein are contemplated herein, as well as racemates.

As used herein, the term “salts” refer to derivatives of the disclosed compounds wherein the parent compound is modified by making acid or base salts thereof. Examples of salts include alkali salts and alkali earth salts of an ionized form of the disclosed compounds. For example, a lithium salt, sodium salt, potassium salt, calcium salt, or magnesium salt. The disclosed compounds may be a salt comprising a cationic metal and an anionic organic compound, for example, a compound having an oxyanion and a sodium cation. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “0.1% to 5%” or “0.1% to 5%” should be interpreted to include not just as 0.1% to 5%, but also the individual values (for example, 1%, 2%, 3%, and 4%) and the sub-ranges (for example, 0.1% to 0.5%, 1.1% to 2.2%, 3.3% to 4.4%) within the indicated range. In addition, the term “in the range” or “within a range” (and similar statements) includes the endpoints of the stated range. Herein, for example, “up to” a number (for example, “up to 50”) includes the number (for example, 50).

In this document, the terms “a,” “an,” or “the” are used to include one or more than one unless the context clearly dictates otherwise. The term “or” is used to refer to a nonexclusive “or” unless otherwise indicated. Further, term “and/or” means one or all of the listed elements or a combination of any two or more of the listed elements.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps may be carried out in any order without departing from the principles of the invention, except when a temporal or operational sequence is explicitly recited. Furthermore, specified steps may be carried out concurrently unless explicit claim language recites that they be carried out separately. For example, a claimed step of doing X and a claimed step of doing Y may be conducted simultaneously within a single operation, and the resulting process will fall within the literal scope of the claimed process.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and may include modification thereto and permutations thereof.

### EXAMPLES

The present invention can be better understood by reference to the following examples which are offered by way of illustration. The present invention is not limited to the examples given herein.

### General Methods

Various compounds of the disclosure can be synthesized according to various methods including, but not limited to, the synthetic approaches described in PCT International Application WO1996/015122 A1, U.S. Pat. No. 4,962,192, or U.S. Pat. No. 5,004,565.

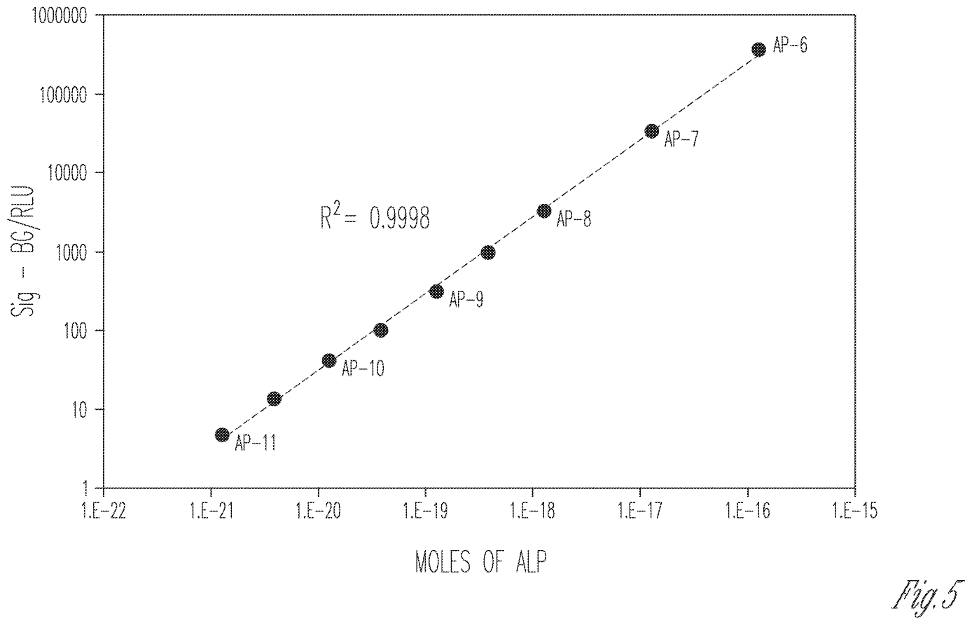

Chemiluminescence (emission) intensity can be measured using a Turner Designs (Sunnyvale, CA) model TD-20e luminometer, a BMG Labtech luminescence plate reader, or a charge-coupled device (CCD) camera luminometer, or any other suitable light intensity measuring devices. In the examples listed below, solutions containing alkaline phosphatase at different concentrations were used (for example, AP4, having an ALP concentration of 1.2×10⁻¹⁵ mol/μL; AP6, having an ALP concentration of 1.2×10⁻¹⁷ mol/μL; AP8, having an ALP concentration of 1.2×10⁻¹⁹ mol/μL; and AP9 having an ALP concentration of 1.2×10⁻²⁰ mol/μL). The compounds and surfactants were tested at their approximately optimal concentrations. As seen in FIG. 5, the RLU for VPPD in a polymeric phosphonium surfactant as a function of ALP concentration is substantially linear (100 μL of the initial solution combined with a solution of 10 μL ALP, data collected over 240 seconds).

Nuclear magnetic resonance (NMR) spectra were obtained using a 400 MHz spectrometer in solutions of D₂O and CDCl₃.

The amine-based buffer “221” or “Sigma-221” can be obtained from Sigma-Aldrich (St. Louis, MO). But other commercially available buffers can be used in the compositions described herein.

### Example 1

A dioxetane compound [Lumigen® PPD] was obtained from Lumigen, Inc., having the structure:

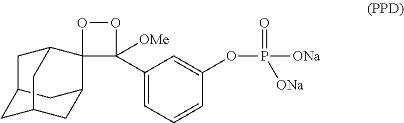

the synthesis of PPD is described in WO2021/086977 (Example 12).

¹H NMR (400 MHz, D₂O) δ ppm 7.40-7.15 (m, 4H), 3.24 (s, 3H), 2.89 (s, 1H), 2.28 (s, 1H), 1.90-1.50 (m, 10H), 1.28 (d, J=13.2 Hz, 1H), 0.99 (d, J=10 Hz, 1H).

### Example 2

VPPD, having the structure:

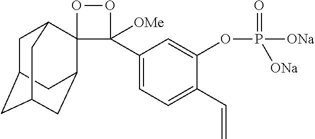

was obtained using the methods described in WO2021/086977 (Example 16).

¹H NMR (400 MHz, D₂O) δ ppm 7.67 (d, J=8.4 Hz, 1H), 7.66 (br, 1H), 7.26 (br, 1H), 7.16-6.06 (m, 1H), 5.88 (d, J=16 Hz, 1H), 5.37 (d, J=12.4 Hz, 1H), 3.27 (s, 3H), 2.88 (s, 1H), 2.30 (s, 1H), 1.84-1.56 (m, 10H), 1.28 (d, J=9.2 Hz, 1H), 1.05 (d, J=12.8 Hz, 1H).

### Example 3

VMPD, having the structure:

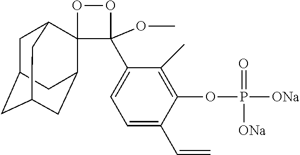

was obtained using the methods described in WO2021/086977 (Example 18).

¹H NMR (400 MHz, D₂O) δ ppm (D₂O ppm): 7.51-7.41 (m, 2H), 7.29-7.20 (m, 1H), 5.66 (d, J=17.2 Hz, 1H), 5.19 (d, J=11.2 Hz, 1H), 3.07 (s, 3H), 2.72 (s, 1H), 2.36 (s, 3H), 1.99 (s, 1H), 1.95-1.38 (m, 10H), 1.19-1.15 (m, 1H), 0.92-0.88 (m, 1H).

### Example 4

An initial solution of the dioxetane compounds of Examples 1-3 and surfactant was prepared in a suitable buffer (for example, 221 buffer). For each example, the dioxetane concentration used was a concentration within the range of 50 mg/L to 500 mg/L. FIG. 2B, further discussed below, shows varying concentrations of the dioxetane VPPD (within the range 50 mg/L to 500 mg/L) while keeping the surfactant EXL concentration constant. The surfactant concentration used was a concentration in the range of 100 mg/L to 500 mg/L. FIG. 2C, further discussed below, shows varying concentrations of the surfactant EXL (within the range 100 mg/L to 500 mg/L) while keeping the concentration of the dioxetane VPPD constant. The ratio of the concentration of dioxetane compound to surfactant was a single ratio in a range of 5:1 to 1:10. In one example, the dioxetane concentration (for example VPPD) was a concentration from 100 mg/L to 150 mg/L; the surfactant concentration (for example, EXL) was a concentration from 200 mg/L to 300 mg/L; the ratio of the concentration of dioxetane (for example, VPPD) to surfactant (for example, EXL) was a ratio in the range of 1:1 to 1:3. The compositions tested comprised from 50 mg/L to 500 mg/L magnesium (II) chloride (MgCl₂), though other magnesium (II) salts can be used.

Next, 100 μL of the initial solution was combined with a solution of 10 μL of an alkaline phosphatase (AP8) at 37° C. The intensity of chemiluminescence was measured over time upon combining the compound with the alkaline phosphatase solution. A graph showing the intensity of light emission (relative luminescence units (RLU) over time) specifically for the 100 μL of initial solution combined with a solution of 10 μL of AP8 is provided at FIGS. 1 and 2A, with FIG. 1 showing the RLU data as a function of time for LP530. The fluorescein surfactant in LP530 has the formula:

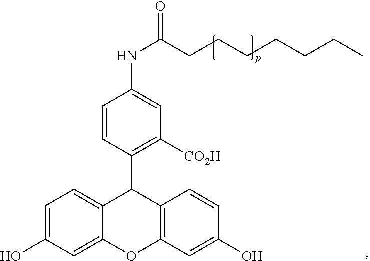

wherein p is an integer from 2 to 10, such as from 5 to 9 or 5 to 7; in combination with cetyltrimethylammonium bromide (CTAB).

In a separate experiment, EXL surfactant was used. A graph showing the intensity of light emission (relative luminescence units (RLU) over time is provided at FIG. 2A. The EXL surfactant comprising repeating unit (A), repeating unit (B) or both:

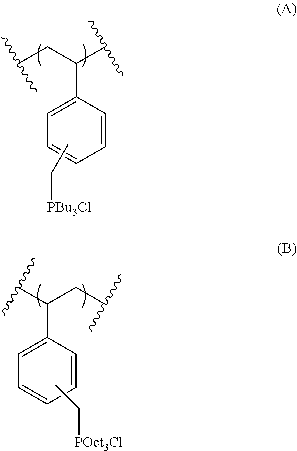

wherein “Bu₃” refers to “tributyl, “Oct₃” refers to “trioctyl.” The molar ratio of the repeating unit (A) to repeating unit (B) used was 4:1. FIGS. 2B and 2C are plots of RLU over time at various VPPD concentrations (50 mg/L to 500 mg/L) and various EXL surfactant concentrations (100 mg/L to 500 mg/L). In both experiments the solution of VPPD and surfactant was mixed with 6 μL AP8 and then incubated at 37° C. FIGS. 2B and 2C show that varying concentrations of the dioxetane VPPD (while keeping the surfactant EXL concentration constant) or varying concentrations of the surfactant EXL (while keeping the dioxetane VPPD concentration constant) provide improved luminescence (as measured in RLUs) compared to EXL and PPD. Note, moreover, that 6 μL AP8 was used in FIGS. 2B and 2C and 10 μL of AP8 was used in FIG. 2A. With 10 μL of AP8 (not shown), the RLUs observed were higher, varied even less, and were even more improved relative to EXL and PPD than with 6 μL AP8.

FIG. 3 shows a comparison of the luminesence observed for VPPD using EXL surfactant compared to VPPD, PPD, and LP530, normalized to the intensity of VPPD in EXL surfactant.

One observation from FIGS. 1, 2A-2C, and 3 is that, relative to PPD and VMPD, VPPD exhibits a higher RLU in a relatively short period of time (e.g., approximately 4 minutes) regardless of the surfactant system (LP530 vs. EXL surfactant). But FIG. 3 accentuates the dramatic relative difference in brightness in RLU between VPPD in EXL compared to LP530. For example, VPPD's brightness is over 20 times higher in EXL relative to LP530 (compare ~9,500 RLU in EXL surfactant vs. ~380 in LP530). One benefit of the compositions described herein is that one can improve alkaline phosphatase detection limits. With the compositions described herein, one can not only detect the ALP in AP10 (1.2×10⁻²¹ mol/μL ALP), but also the ALP in AP11 and AP12 (1.2×10⁻²² mol/μL and 1.2×10⁻²³ mol/μL ALP concentration, respectively).

### Example 5

Dioxetane compositions comprising the dioxetane concentrations, surfactant/enhancer concentrations, oxetane:surfactant ratios, and magnesium amounts described in Example 4 were prepared using the following surfactants/enhancers (1)-(4):

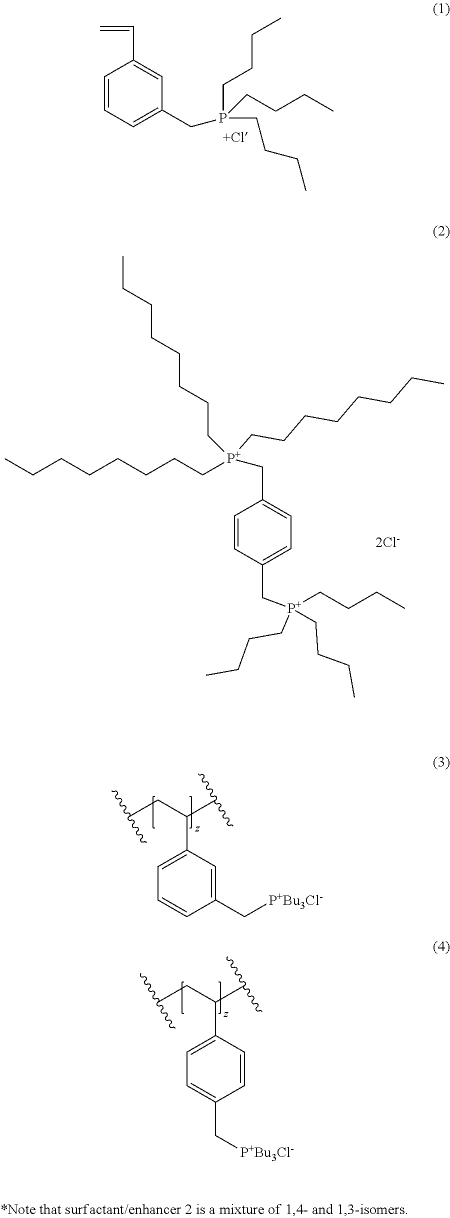

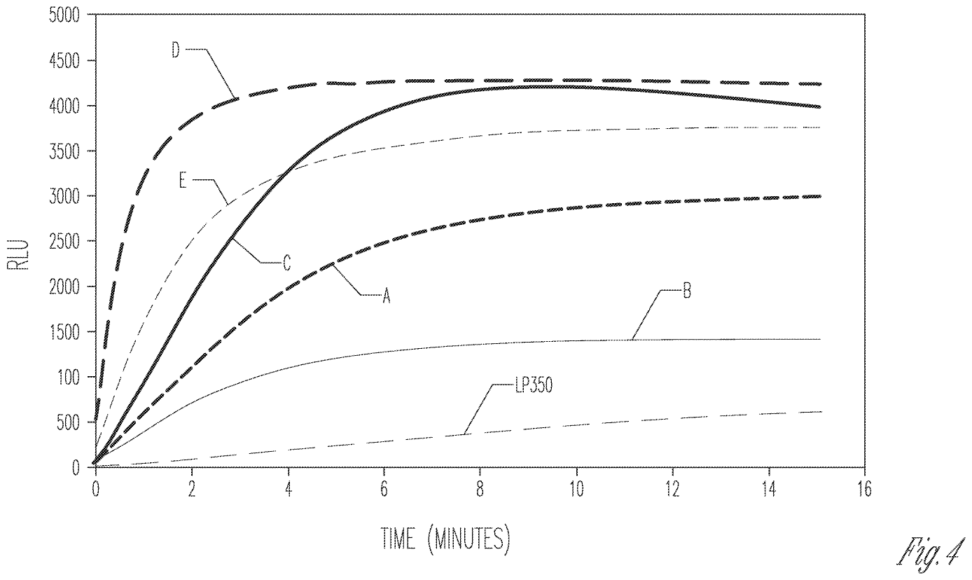

The oxetane and enhancer combinations used to generate the data in FIG. 4 is listed below in Table 1.

| | Oxetane | Enhancer |
|---|---|---|
| A | VPPD | (1) |
| B | VPPD | (1) |
| C | VPPD | (2) |
| D | VPPD | (3) |
| E | VPPD | (4) |
| F | PPD | Fluorescein |

FIG. 4 shows a comparison of the luminesence observed for VPPD using surfactants/enhancers (1)-(4) when mixed with AP8 compared to LP530. Specifically, 100 μL of a solution including VPPD and surfactants/enhancers was combined with 10 μL of AP8 and then incubated at 37° C.

One observation from FIG. 4 is that VPPD in phosphonium surfactants/enhancers, regardless of whether they are polymeric or small molecule phosphonium surfactants/enhancers, gave significantly higher RLU in a relatively short period of time (e.g., approximately 4 minutes) relative to LP530. Another observation is that small molecule phosphonium surfactant (2) and polymeric phosphonium surfactant/enhancer (4) have about the same RLU at 4 minutes. And there seems to be a difference in the RLU at 4 minutes (and beyond) between the isomeric polymeric surfactants/enhancers (3) and (4), where (3) is brighter than (4).

What is claimed is:

1. A composition comprising:
a compound of Formula I and salts thereof: 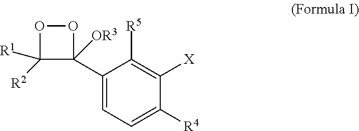
wherein each of R¹ and R² is independently C₃-C₁₀ alkyl, or R¹ and R² taken together with the carbon to which they are attached provide a C₅-C₁₀ cycloalkyl ring;
R³ is C₁-C₁₀ alkyl, C₆-C₁₀ aryl, or heteroaryl;
R⁴ is C₂-C₁₀ alkenyl;
R⁵ is H or C₁-C₁₀ alkyl; and
X is a phosphate; and at least one phosphonium surfactant.

2. The composition of claim 1, wherein the composition comprises one surfactant.

3. The composition of claim 1, wherein the composition comprises two or more surfactants.

4. The composition of claim 1 or 3, wherein the phosphonium surfactant is selected from the group consisting of small-molecule phosphonium surfactants and polymeric phosphonium surfactants.

5. The composition of claim 1 or 4, wherein the phosphonium surfactant comprises a polymeric phosphonium surfactant comprising repeating unit (A), repeating unit (B) or both: 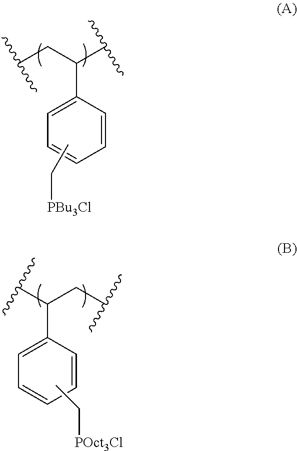
wherein “Bu₃” refers to “tributyl and “Oct₃” refers to “trioctyl.”

6. The composition of claim 5, wherein a molar ratio of the repeating unit (A) to repeating unit (B) is from 1:100 to 100:1.

7. The composition of claim 1 or 4, wherein the polymeric phosphonium surfactant comprises the structure: 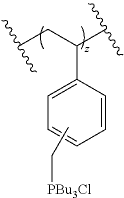
wherein z is an integer from 2 to 1000 or from 100 to 600.

8. The composition of claim 1 or 4, wherein the polymeric phosphonium surfactant comprises a small molecule phosphonium surfactant of the formula: 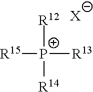
wherein:
R¹²-R¹⁴ are each, independently, C₁-C₁₀ alkyl;
R¹⁵ is arylalkyl; and
X⁻ is a counterion.

9. The composition of claim 8, wherein R¹⁵ is arylalkyl substituted with a C₂-C₈ alkenyl group or with a group of the formula: 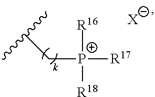
wherein k is 0, 1, or 2; R¹⁶-R¹⁸ are each, independently, C₁-C₁₆ alkyl.

10. The composition of claim 9, wherein R¹⁶-R¹⁸ are each, independently, C₄-C₁₀ alkyl.

11. The composition of claim 9, wherein the C₂-C₈ alkenyl group is a group of the formula: 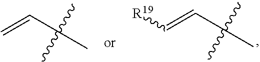
wherein R¹⁹ is C₁-C₈ alkyl, C₂-C₈ alkenyl or C₂-C₈ alkynyl.

12. The composition of claim 1, wherein the composition further comprises a fluorescein surfactant.

13. The composition of claim 12, wherein the fluorescein surfactant has the formula (V): 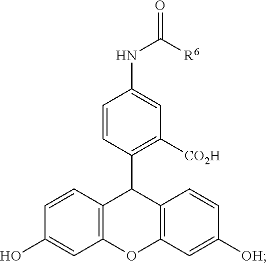
wherein R⁶ is a C₆-C₂₀-alkyl group and optionally comprises an ammonium salt, such as cetyltrimethylammonium bromide; and optionally, wherein the fluorescein surfactant is 5-dodecanoylaminofluorescein, 5-hexadecanoylaminofluorescein or 5-stearylaminofluorescein.

14. The composition of claim 1, wherein the phosphate group is of the formula —O—P(O)(ONa)(ONa).

15. The composition of claim 1, wherein the compound of the Formula I is a compound of Formula II, or a salt thereof: 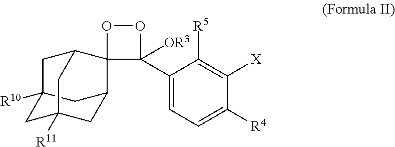
wherein each of R¹⁰ and R¹¹ is independently H, halogen, C₁-C₁₀ alkyl, C₂-C₁₀ alkenyl, C₆-C₁₀ aryl.

16. The composition of claim 15, wherein R¹⁰ and R¹¹ are each independently H or halogen.

17. The composition of any of claims 1-16, wherein the compound of Formula I is a compound of Formula III, or a salt thereof:

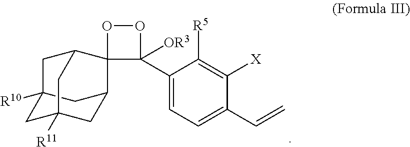

18. The composition of any of claims 1-16 wherein the compound of Formula I is a compound of Formula IV, or a salt thereof:

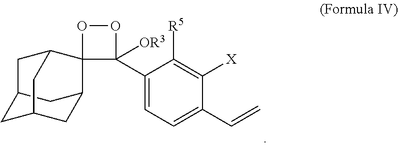

19. The composition of any of claims 1-16, wherein the compound of Formula I is a compound of the formula: 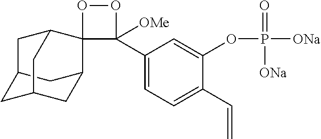
or a salt thereof.

20. The composition of any of claims 1-16, wherein the compound of Formula I is a compound of the formula: 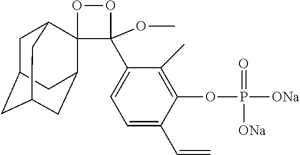
or a salt thereof.

21. The composition of claim any preceding claim further comprising an amine buffer.

22. The composition of any preceding claim in the form of an aqueous composition.

23. The composition of any preceding claim, wherein the composition comprises alkaline phosphatase and wherein the alkaline phosphatase has a concentration in the composition of at most 1×10⁻¹⁸ mol/μL, 1×10⁻¹⁹ mol/μL, 1×10⁻²⁰ mol/μL, or 1×10⁻²¹ mol/μL.

24. The composition of claim 23, wherein the alkaline phosphatase has a concentration in the composition of at least 1.2×10⁻²² mol/μL or at least 1.2×10⁻²³ mol/μL.

25. The composition of claim 23 or 24, wherein the composition comprises at least 1×10⁻²² or at least 1×10⁻²¹ mol ALP.

26. The composition of claim 23, 24 or 25 wherein the composition comprises at most 1×10⁻¹⁷ mol ALP, at most 1×10⁻¹⁸ mol ALP, at most 1×10⁻¹⁹ mol ALP, or at most 1×10⁻²⁰ mol ALP.

27. A method of detecting an alkaline phosphatase in a sample, comprising contacting the sample with a composition of any preceding claim thereby producing a mixture and monitoring the mixture for luminescence.

28. The method of claim 27, wherein the alkaline phosphatase in the mixture has a concentration of at most 1×10⁻¹⁸ mol/μL, at most 1×10⁻¹⁹ mol/μL, at most 1×10⁻²⁰ mol/μL, or at most 1×10⁻²¹ mol/μL.

29. The method of claim 27 or 28, wherein the alkaline phosphatase in the mixture has a concentration of at least 1×10⁻²² mol/μL or at least 1×10⁻²³ mol/μL.

30. The method of any of claims 27, 28 or 29, wherein the mixture comprises at most 1×10⁻¹⁷ mol ALP, at most 1×10⁻¹⁸ mol ALP, at most 1×10⁻¹⁹ mol ALP, or at most 1×10⁻²⁰ mol ALP.

31. The method of any of claims 27, 28, 29 or 30, wherein the mixture comprises at least 1×10⁻²¹ mol ALP or at least 1×10⁻²² mol ALP.

32. The method of any of claims 27, 28, 29, 30 or 31, further comprising measuring the intensity of a resulting luminescence and correlating the intensity with a presence of the alkaline phosphatase or with a concentration of the alkaline phosphatase.

33. The method of any of claims 27-32, wherein the luminescence has at least one of a peak RLU of greater than 8,000; and a T_{1/2} of 3 minutes or less.

34. The method of any of claims 27-33, wherein the sample is an aqueous sample having a pH of 4 to 12.

35. The method of claim 27, wherein the sample is an aqueous sample having a pH of 8 to 12.

36. The method of any of claims 27-35, wherein the method comprises contacting the sample with a composition comprising the phosphonium surfactant and wherein the method has an increased signal-to-noise ratio when compared with contacting the sample with a composition comprising PPD and a fluorescein surfactant having the formula: 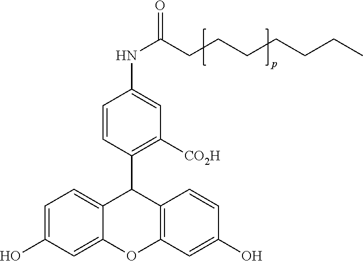
wherein p is an integer from 2 to 10, such as from 5 to 9 or 5 to 7; in combination with cetyltrimethylammonium bromide (CTAB).

37. The method of claim 36, wherein the increased signal-to-noise ratio is at least 50-fold.

38. The method of claim 36, wherein a luminescence of the composition comprising the phosphonium surfactant in the absence of the sample has an RLU of less than 15.

39. The method of claim 36, wherein a luminescence of the composition comprising the phosphonium surfactant in the absence of the sample has an RLU of greater than 1 and less than 15.

40. A kit for determining the presence of an alkaline phosphatase, the kit comprising a compound of Formula I and salts thereof: 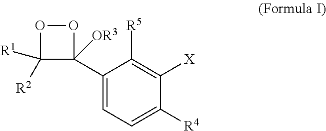
wherein each of R¹ and R² is independently C₃-C₁₀ alkyl, or R¹ and R² taken together with the carbon to which they are attached provide a C₅-C₁₀ cycloalkyl ring;
R³ is C₁-C₁₀ alkyl, C₆-C₁₀ aryl, or heteroaryl;
R⁴ is C₂-C₁₀ alkenyl;
R⁵ is H or C₁-C₁₀ alkyl; and; and
at least one phosphonium surfactant; and optionally, wherein the composition comprises one surfactant.

41. The kit of claim 40, wherein the phosphonium surfactant is selected from the group consisting of small-molecule phosphonium surfactants and polymeric phosphonium surfactants.

42. The kit of claim 40 or 41, wherein the polymeric phosphonium surfactant comprises repeating unit (A), repeating unit (B) or both: 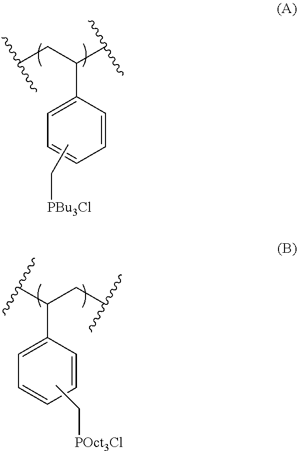
wherein “Bu₃” refers to “tributyl and “Oct₃” refers to “trioctyl.”

43. The kit of claim 42, wherein a molar ratio of the repeating unit (A) to repeating unit (B) is from 1:100 to 100:1.

44. The kit of claim 40 or 41, wherein the polymeric phosphonium surfactant comprises the structure: 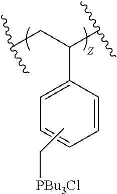
wherein z is an integer from 2 to 1000 or from 100 to 600.

45. The kit of claim 40 or 41, wherein the small molecule phosphonium surfactant is of the formula: 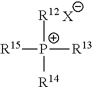
wherein:
R¹²-R¹⁴ are each, independently, C₁-C₁₀ alkyl;
R¹⁵ is an arylalkyl that is optionally substituted; and
X⁻ is a counterion.

46. The kit of claim 45, wherein R¹⁵ is arylalkyl substituted with a C₂-C₈ alkenyl group or with a group of the formula: 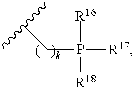
wherein k is 0, 1, or 2; R¹⁶-R¹⁸ are each, independently, C₁-C₁₆ alkyl.

47. The kit of claim 46, wherein R¹⁶-R¹⁸ are each, independently, C₄-C₁₀ alkyl.

48. The kit of claim 46, wherein the C₂-C₈ alkenyl group is a group of the formula: 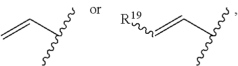
wherein R¹⁹ is C₁-C₈ alkyl, C₂-C₈ alkenyl or C₂-C₈ alkynyl.

49. The kit of claim 40, wherein the kit further comprises a fluorescein surfactant wherein the fluorescein surfactant has the formula (V): 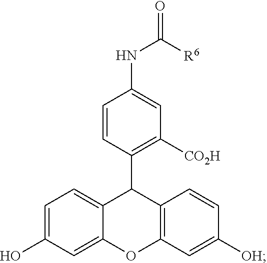
wherein R⁶ is a C₆-C₂₀-alkyl group and optionally comprises an ammonium salt, such as cetyltrimethylammonium bromide; and optionally, wherein the fluorescein surfactant is 5-dodecanoylaminofluorescein, 5-hexadecanoylaminofluorescein or 5-stearylaminofluorescein.

50. The kit of claim 49, wherein the surfactant is 5-dodecanoylaminofluorescein, 5-hexadecanoylaminofluorescein or 5-stearylaminofluorescein.

51. The kit of any of claims 40-50, wherein the compound of Formula I and salts thereof; and the at least one surfactant are in separate containers.

52. The kit of any of claims 40-50, wherein the compound of Formula I and salts thereof; and the at least one surfactant are in a single container.

53. The composition, method, or kit of any preceding claim, wherein the dioxetane concentration is from 100 mg/L to 150 mg/L.

54. The composition, method, or kit of any preceding claim, wherein the surfactant concentration is from 200 mg/L to 300 mg/L.

55. The composition, method, or kit of any preceding claim, wherein the ratio of dioxetane to surfactant is from 1:1 to 1:3.

56. The composition, method, or kit of any preceding claim, wherein the composition comprises from 50 mg/L to 500 mg/L of a magnesium (II) salt.

57. The composition, method, or kit of any preceding claim, wherein the composition comprises from 50 mg/L to 500 mg/L of MgCl₂.

58. A method of detecting an alkaline phosphatase in a sample, comprising:
a. providing a mixture of a dioxetane compound and a phosphonium surfactant;
b. contacting the sample with the mixture of the dioxetane compound and the phosphonium surfactant to produce a mixture comprising the sample; and
c. monitoring luminescence of the mixture comprising the sample, wherein the monitoring the luminescence comprises detecting a peak RLU greater than 8,000 or a T_{1/2} of 3 minutes or less.

59. The method of claim 58, wherein a luminescence of a combination of the dioxetane compound and the phosphonium surfactant in the absence of the sample has a peak RLU less than 15.

60. The method of claim 58, wherein a luminescence of a combination of the dioxetane compound and the phosphonium surfactant in the absence of the sample has a peak RLU greater than 1 and less than 15.

61. The method of any of claims 58-60, wherein the alkaline phosphatase in the mixture comprising the sample has a concentration of at most 1×10⁻¹⁸ mol/μL, 1×10⁻¹⁹ mol/μL, 1×10⁻²⁰ mol/μL, or 1×10⁻²¹ mol/μL.

62. The method of any of claims 58-61, wherein the alkaline phosphatase in the mixture comprising the sample has a concentration of at least 1×10⁻²² mol/μL or 1×10⁻²³ mol/μL.

63. The method of any of claims 58-62, wherein the mixture comprising the sample comprises at most 1×10⁻¹⁷ mol, 1×10⁻¹⁸ mol, 1×10⁻¹⁹ mol, or 1×10⁻²⁰ mol ALP.

64. The method of any of claims 58-63, wherein the mixture comprises at least 1×10⁻²¹ mol or 1×10⁻²² mol ALP.

65. A method of detecting an alkaline phosphatase in a sample, comprising:
a. contacting the sample with a first mixture of a dioxetane compound and a phosphonium surfactant to produce a second mixture comprising the sample; and
b. monitoring luminescence of the second mixture, wherein the monitoring the luminescence comprises detecting a peak RLU greater than 8,000 or a T_{1/2} of 3 minutes or less.

66. The method of claim 65, wherein a luminescence of a combination of the dioxetane compound and the phosphonium surfactant in the absence of the sample has a peak RLU less than 15.

67. The method of claim 65, wherein a luminescence of a combination of the dioxetane compound and the phosphonium surfactant in the absence of the sample has a peak RLU greater than 1 and less than 15.

68. The method of any of claims 65-67, wherein the alkaline phosphatase in the second mixture has a concentration of at most 1×10⁻¹⁸ mol/μL, 1×10⁻¹⁹ mol/μL, 1×10⁻²⁰ mol/μL, or 1×10⁻²¹ mol/μL.

69. The method of any of claims 65-68, wherein the alkaline phosphatase in the second mixture has a concentration of at least 1×10⁻²² mol/μL or 1×10⁻²³ mol/μL.

70. The method of any of claims 65-69, wherein the second mixture comprises at most 1×10⁻¹⁷ mol, 1×10⁻¹⁸ mol, 1×10⁻¹⁹ mol, or 1×10⁻²⁰ mol ALP.

71. The method of any of claims 65-70, wherein the second mixture comprises at least 1×10⁻²¹ mol or 1×10⁻²² mol ALP.

72. The method of any of claims 65-70, wherein the second mixture comprises
at least 10 μL, at least 50 μL, at least 100 μL; and/or
up to 100 μL, up to 110 μL, up to 150 μL, up to 200 μL, or up to 500 μL.
